(19) United States
(12) Patent Application Publication
(10) Pub. No.: US 2007/0149605 A1
(43) Pub. Date: Jun. 28, 2007

(54) Substituted pyrrole derivatives as hmg-coa reductase inhibitors

(76) Inventors: Jitendra Sattigeri, Haryana (IN); Mohammad Salman, Plainsboro, NJ (US); Yatendra Kumar, Haryana (IN)

Correspondence Address: RANBAXY INC., 600 COLLEGE ROAD EAST, SUITE 2100, PRINCETON, NJ, 08540, US

(21) Appl. No.: 10/558,858

(22) PCT Filed: May 28, 2004
(86) PCT No.: PCT/IB04/01754

§ 371 Date: Oct. 31, 2006

Related U.S. Application Data
(63) Continuation-in-part of application No. 10/448,770, filed on May 30, 2003, now abandoned.

(51) Int. Cl. A61K 31/4025; A61K 31/401; C07D 405/02

(52) U.S. Cl. 514/422; 514/423; 548/517; 548/537

(57) ABSTRACT

The present invention relates to substituted pyrrole derivatives of Formula (I), wherein (Y), with the proviso that one of R₂, R₄ and R₅ is a heterocycle and with the further provision that if R₂ is not a heterocycle then either R₄ or R₅ alone is not unsubstituted pyridyl, which can be used as 3-hydroxy-3-methylglutaryl-coenzyme A (HMG-CoA) reductase inhibitors. Compounds disclosed herein can function as cholesterol lowering agents and can be used for the treatment of cholesterol-related diseases and related symptoms. Processes for the preparation of disclosed compounds are provided, as well as pharmaceutical compositions containing the disclosed compounds, and methods of treating cholesterol-related diseases and related symptoms.

### FIELD OF THE INVENTION

The present invention relates to substituted pyrrole derivatives, which can be used as 3-hydroxy-3-methylglutaryl-coenzyme A (HMG-CoA) reductase inhibitors. Compounds disclosed herein can function as cholesterol lowering agents and can be used for the treatment of cholesterol-related diseases and related symptoms. Processes for the preparation of disclosed compounds are provided, as well as pharmaceutical compositions containing the disclosed compounds, and methods of treating cholesterol-related diseases and related symptoms.

### BACKGROUND OF THE INVENTION

Cardiovascular disease and its associated maladies, dysfunctions and complications are a principal cause of disability and the chief cause of death. One specific factor significantly contributing to this pathophysiologic process is atherosclerosis, which has been generally recognized as the leading health care problem both with respect to mortality and health care costs.

Atherosclerosis is characterized by the deposition of fatty substances, primarily cholesterol, resulting in plaque formation on the inner surface of the arterial wall and degenerative change to the arteries.

It is now well established that cardiovascular disorders including myocardial infarction, coronary heart disease, hypertension and hypotension, cerebrovascular disorders including stroke, cerebral thrombosis and memory loss due to stroke; peripheral vascular disease and intestinal infarction are caused by blockage of arteries and arterioles by atherosclerotic plaque. Atherosclerotic plaque formation is multi-factorial in its production. Hypercholesterolemia, especially elevated levels of low-density lipoprotein cholesterol (LDL), is an important risk factor for atherosclerosis and arteriosclerosis and associated diseases.

The HMG-CoA reductase inhibitors (statins) have been used in reducing blood levels of LDL cholesterol. Cholesterol is produced via the mevalonic acid pathway. Reducing the formation of mevalonic acid, a precursor to cholesterol, leads to a corresponding decrease in hepatic cholesterol biosynthesis with a reduction in the cellular pool of cholesterol.

U.S. Pat. No. 4,681,893 assigned to Warner-Lambert, discloses certain trans-6-[2-(3-, or 4-carbaoxamido-substituted pyrrole-1-yl)alkyl]-4-hydroxypyran-2-ones and the corresponding ring-opened hydroxy acids derived therefrom, including trans(±)-5-(4-fluorophenyl)-2-(1-methylethyl)-N,4-diphenyl-1-[2-tetrahydro-4-hydroxy-6-oxo-2H-pyran-2-yl)ethyl]-1H-pyrrole-3-carboxamide, which are inhibitors of 3-hydroxy-3-methylglutaryl-coenzyme A reductase (HMG-CoA), an important coenzyme catalyzing the intracellular synthesis of cholesterol.

U.S. Pat. No. 5,273,995 assigned to Warner Lambert, relates to the optically pure (R, R) form of the ring-opened acid of trans-5-(4-fluorophenyl)-2-(1-methylethyl-N,4-diphenyl-1-[2-tetrahydro-4-hydroxy-6-oxo-2H-pyran-2-yl)ethyl]-1H-pyrrole-3-carboxamide that is [R-(R*, R*)]-2-(4-fluorophenyl)-β,δ-dihydroxy-5-(1-methylethyl)-3-phenyl-4-[(phenylamino)carbonyl]-1H-pyrrole-1-heptanoic acid, pharmaceutically acceptable salts thereof, specifically its calcium salt (Atorvastatin, Lipitor®), which is currently being used for the treatment of hypercholesterolemia.

U.S. Pat. No. 5,385,929 discloses certain phenyl hydroxy derivatives of the compounds disclosed in U.S. Pat. No. 5,273,995, and that such phenyl hydroxy derivatives are also active as the inhibitors of the biosynthesis of cholesterol.

### SUMMARY OF THE INVENTION

The present invention relates to substituted pyrrole derivatives, which can be used as 3-hydroxy-3-methylglutaryl-coenzyme A (HMG-CoA) reductase inhibitors, and a process for the synthesis of these compounds.

Pharmaceutical composition containing the compounds, and which may also contain pharmaceutically acceptable carriers or diluents, which can be used for the treatment of cholesterol-related disease or related symptoms thereof are also provided.

Other aspects will be set forth in the accompanying description which follows and in the part will be apparent from the description or may be learnt by the practice of the invention.

In accordance with one aspect, there are provided compounds having the structure of Formula I, 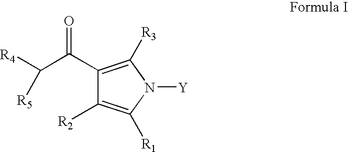
their pharmaceutically acceptable salts, pharmaceutically acceptable solvates, tautomers, racemates, polymorphs, pure enantiomers, diastereoisomers, metabolites, prodrugs or N-oxides wherein 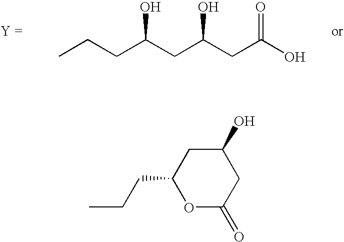
R₁ can be C₁-C₆ alkyl, C₃-C₆ cycloalkyl, or optionally substituted phenyl, wherein up to three substituents are independently selected from [halogens, C₁-C₆ alkyl, hydroxyl, C₁-C₃ alkoxy, protected hydroxyl, carboxyl, acetyl, optionally substituted amino wherein up to two substituents are independently selected from C₁-C₆ alkyl, C₃-C₆ cycloalkyl, SO₂R₆, COR₆, CONHR₆ (wherein R₆ is C₁-C₆ alkyl or aryl), C₁-C₃ alkoxycarbonyl, cyano and C₁-C₃ perfluoroalkyl].
R₃ can be optionally substituted C₁-C₆ alkyl or C₃-C₆ cycloalkyl (wherein the substituents are selected halogens, hydroxyl, C₁-C₃ alkoxy, and protected hydroxyl); or —NR₇R₈ wherein R₇ and R₈ are optionally substituted C₁-C₆ alkyl (wherein the optional substituent(s) is/are selected from halogens, hydroxyl. C₁-C₃ alkoxy, and protected hydroxyl).
R₂, R₄ and R₅ can be independently selected from: hydrogen, C₁-C₆ alkyl, C₃-C₆ cycloalkyl, aralkyl, optionally substituted aryl (wherein the substituents are selected from C₁-C₆ alkyl, C₁-C₆ carbonyl alkyl, C₁-C₆ hydroxyalkyl, halogens, cyano, hydroxyl, protected hydroxyl, C₁-C₆ alkoxy, C₁-C₃ perfluoroalkyl, SO₂NHR₆ (wherein R₆ is C₁-C₆ 3alky, or aryl), COOR₆ wherein R₆ is C₁-C₆ alkyl, or aryl, and —NR₇R₈ wherein R₇ and R₈ are selected from {hydrogen, optionally substituted C₁-C₆ alkyl [wherein the optional substituent(s) is/are selected from halogens, hydroxyl, C₁-C₃ alkoxy, protected hydroxyl, and cyano] optionally substituted C₃-C₆ cycloalkyl [wherein the optional substituent(s) is/are selected from halogens, hydroxyl, C₁-C₃ alkoxy, protected hydroxyl, and cyano], SO₂R₆, COR₆, CONH₂, CONHR₆, COOR₆ [wherein R₆ is C₁-C₆ alkyl or aryl], and optionally substituted aryl [wherein the optional substituent(s) is/are selected from halogens, C₁-C₃ alkyl, hydroxyl, C₁-C₃ alkoxy, protected hydroxyl, and cyano]} and R₂, R₄ and R₅ can also be optionally substituted heterocycle having one or more hetero atom(s) {wherein said hetero atom(s) is/are selected from oxygen, nitrogen and sulfur, and the optional substituents are selected from [optionally substituted C₁-C₆ alkyl or C₃-C₆ cycloalkyl (wherein the optional substituent(s) is/are selected from halogens, hydroxyl, C₁-C₃ alkoxy, protected hydroxyl, and cyano); halogens, hydroxyl, protected hydroxyl, C₁-C₃ alkoxy, cyano, C₁-C₃ perfluoroalkyl, and optionally substituted aryl (wherein the optional substituents are selected from C₁-C₆ alkyl, halogens, hydroxyl, protected hydroxyl, C₁-C₃ alkoxy, cyano, and C₁-C₃ perfluoroalkyl)]}
   with the proviso that one of R₂, R₄ and R₅ is a heterocycle and with the further provision that if R₂ is not a heterocycle then either R₄ or R₅ alone is not unsubstituted pyridyl.

For example, R₂ can be optionally substituted heterocycle having one or more hetero atom(s) wherein said hetero atom(s) is/are selected from oxygen, nitrogen and sulfur, and the optional substituents are selected from optionally substituted C₁-C₆ alkyl or C₃-C₆ cycloalkyl (wherein the optional substituent(s) is/are selected from halogen, hydroxyl, C₁-C₃ alkoxy, protected hydroxyl and cyano). And for example, R₄ and R₅ can be independently selected from hydrogen, optionally mono or multiple substituted aryl (wherein the substituents are selected from C₁-C₃ carbonyl alkyl, halogen, hydroxyl and C₁-C₃ alkoxy).

As used herein the term “alkyl”, unless otherwise defined, refers to straight or branched chain hydrocarbon of from 1 to 6 carbon atom(s). Examples of alkyl include, but are not limited to, methyl, ethyl, n-propyl, isopropyl, butyl, and the like.

Alkyl may optionally be substituted with halogen, hydroxy, protected hydroxyl, C₁-C₃ alkoxy, optionally substituted amino and C₁-C₆ alkoxycarbonyl.

As used herein the term “alkoxy” stands for a radical represented by Formula O-alkyl wherein alkyl is the same as defined above. Examples of alkoxy include, but are not limited to, methoxy, ethoxy, propoxy, isopropoxy, cyclopentyloxy, and the like.

The term “halogen” as used herein refers to fluorine, chlorine, bromine or iodine. The term “protected hydroxyl” includes, but is not limited to, benzoyl and methylthiomethyl and the like. The term “aryl” as used herein stands for an aromatic radical having 6 to 14 carbon atoms. Examples of aryl include, but are not limited to, phenyl, naphthyl, anthryl and biphenyl, and the like. The term “aralkyl” as used herein stands for an aryl radical having 7 to 14 carbon atoms, which is bonded to an alkylene chain. Examples of aralkyl include, but are not limited to, benzyl, naphthylmethyl, phenethyl and phenylpropyl, and the like. The term “heterocycle” refers to non-aromatic or aromatic ring system having one or more heteroatom(s) wherein the ring system includes mono, bi or tricyclic. Examples of heterocycle include, but are not limited to, thienyl, furyl, pyrrolyl, pyrazolyl, pyridyl, pyrimidyl, pyrazinyl, pyridazinyl, quinolinyl,. isoquinolinyl, quinazolinyl, quinoxalinyl, phthalazinyl, cinnolinyl, thiazolyl, benzthiazolyl, isothiazolyl, oxazolyl, benoxazolyl, isoxazolyl, imidazolyl, benzimidazolyl, pyrazolyl, indolyl, indolinyl and isoindolyl and the like.

In accordance with another aspect, there is provided a method for treating a mammal suffering from cholesterol related disease, diabetes and related disease, cerebrovascular disease or cardiovascular disease, comprising administering to a mammal a therapeutically effective amount of a compound disclosed herein.

The compounds of the present invention can be used for treating arteriosclerosis, atherosclerosis, hypercholesterolemia, hyperlipidemia, hyperlipoproteinemia, hypertriglyceridemia, hypertension, stroke, ischemia, endothelium dysfunction, peripheral vascular disease, peripheral arterial disease, coronary heart disease, myocardial infarction, cerebral infarction, myocardial microvascular disease, dementia, Alzheimer's disease, osteoporosis and/or osteopenia, angina or resterosis. Further compounds which can be useful for treatment of these diseases, and methods for making such compounds, are disclosed in copending U.S. patent application Ser. No. 10/449,418 filed 30 May 2003, entitled “Substituted Pyrrole Derivatives,” and PCT Application No. PCT/IB2004/______ filed ______ entitled “Substituted Pyrrole Derivatives,” which applications are incorporated herein in their entirety.

In accordance with yet another aspect, there are provided process for the preparation of the compounds described herein.

### DETAILED DESCRIPTION OF THE INVENTION

The compounds described herein may be prepared by techniques well known in the art and familiar to the average synthetic organic chemist. In addition, the compounds of the present invention may be prepared by the following reaction sequences as depicted in Schemes I and II.

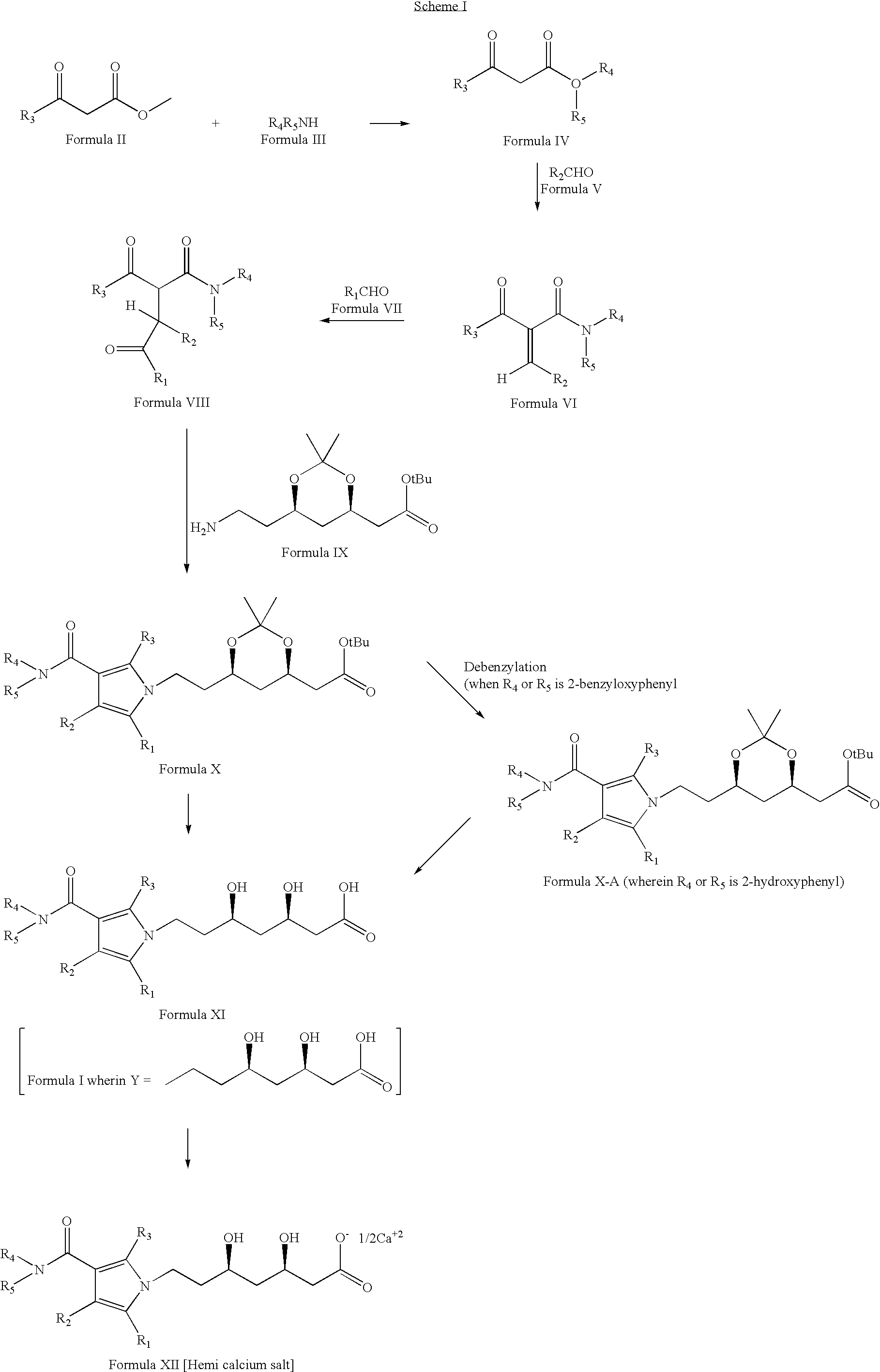

Compounds of Formula XII can be prepared according to Scheme I. Accordingly, a compound of Formula II is reacted with a compound of Formula III (wherein R₃, R₄ and R₅ are as defined earlier) to give a compound of Formula IV, which on reaction with a compound of Formula V (wherein R₂ is as defined earlier) gives a compound of Formula VI, which on treatment with a compound of Formula VII (wherein R₁ is as defined earlier) yields a compound of Formula VIII, which on further reaction with a compound of Formula IX gives a compound of Formula X, which (when R₄ or R₅ is 2-benzyloxyphenyl) on debenzylation gives a compound of Formula X-A (wherein R₄ or R₅ is 2-hydroxyphenyl), the compound of Formula X or X-A on hydrolysis gives a compound of Formula XI, which can be further converted to hemicalcium salt.

The reaction of a compound of Formula II with a compound of Formula III to give a compound of Formula IV can be carried out in a nonpolar solvent, such as xylene or toluene. The reaction of a compound of Formula II with a compound of Formula III can be carried out in the presence of an organic base such as triethylamine, pyridine or 1,2-ethylenediamine.

The reaction of a compound of Formula IV with an aldehyde of Formula V to give a compound of Formula VI can be carried out in a nonpolar solvent, such as hexane, heptane, dichloromethane or toluene. The reaction of a compound of Formula IV with an aldehyde of Formula V can be carried out in the presence of an organic base such as piperidine, pyridine or β-alanine and an organic acid such as glacial acetic acid or benzoic acid.

The reaction of a compound of Formula VI with an aldehyde of Formula VII to give a compound of Formula VIII can be carried out in the presence of a suitable catalyst, such as sodium cyanide, 3-ethyl-5-(2-hydroxyethyl)-4-methyl thiazolium bromide or 3-benzyl-5-(2-hydroxyethyl)-4-methyl thiazolium chloride, in a solvent free condition or in an alcoholic solvent, such as methanol, ethanol, propanol, or isopropanol or ether solvent such as dioxane. The reaction of a compound of Formula VI with an aldehyde of Formula VII can be carried out in the presence of an organic base, such as triethylamine or pyridine.

The reaction of a compound of Formula VIII with a compound of Formula IX to give a compound of Formula X can be carried out in a non polar solvent, such as xylene, hexane, heptane, tetrahydrofuran, toluene or a mixture thereof in a suitable ratio. The reaction of a compound of Formula VIII with a compound of Formula IX can be carried out in the presence of an organic acid, such as pivalic acid or p-toluene sulfonic acid.

The debenzylation of a compound of Formula X to give a compound of Formula X-A can be carried out in the presence of a catalyst, such as palladium on carbon and hydrogen, in a polar solvent, such as methanol, ethanol, propanol or dioxane.

The conversion of a compound of Formula X or X-A to a compound of Formula XI can be carried out in a two-step manner involving an initial acid-catalysed cleavage of ketal, followed by base-catalysed hydrolysis of the tert-butyl ester. The acid can be a mineral acid, such as hydrochloric acid. The cleavage of ketal can be carried out by any other cleavage method known in the prior art. The base can be an inorganic base, such as lithium hydroxide, sodium hydroxide or potassium hydroxide.

The compound of Formula XI can be converted into its corresponding hemi calcium salt by following procedures well-known to a person ordinary skilled in the art. The hemi calcium salts of compound of Formula XI can also be prepared from the corresponding lactone form of Formula XI by following procedures well known in the art.

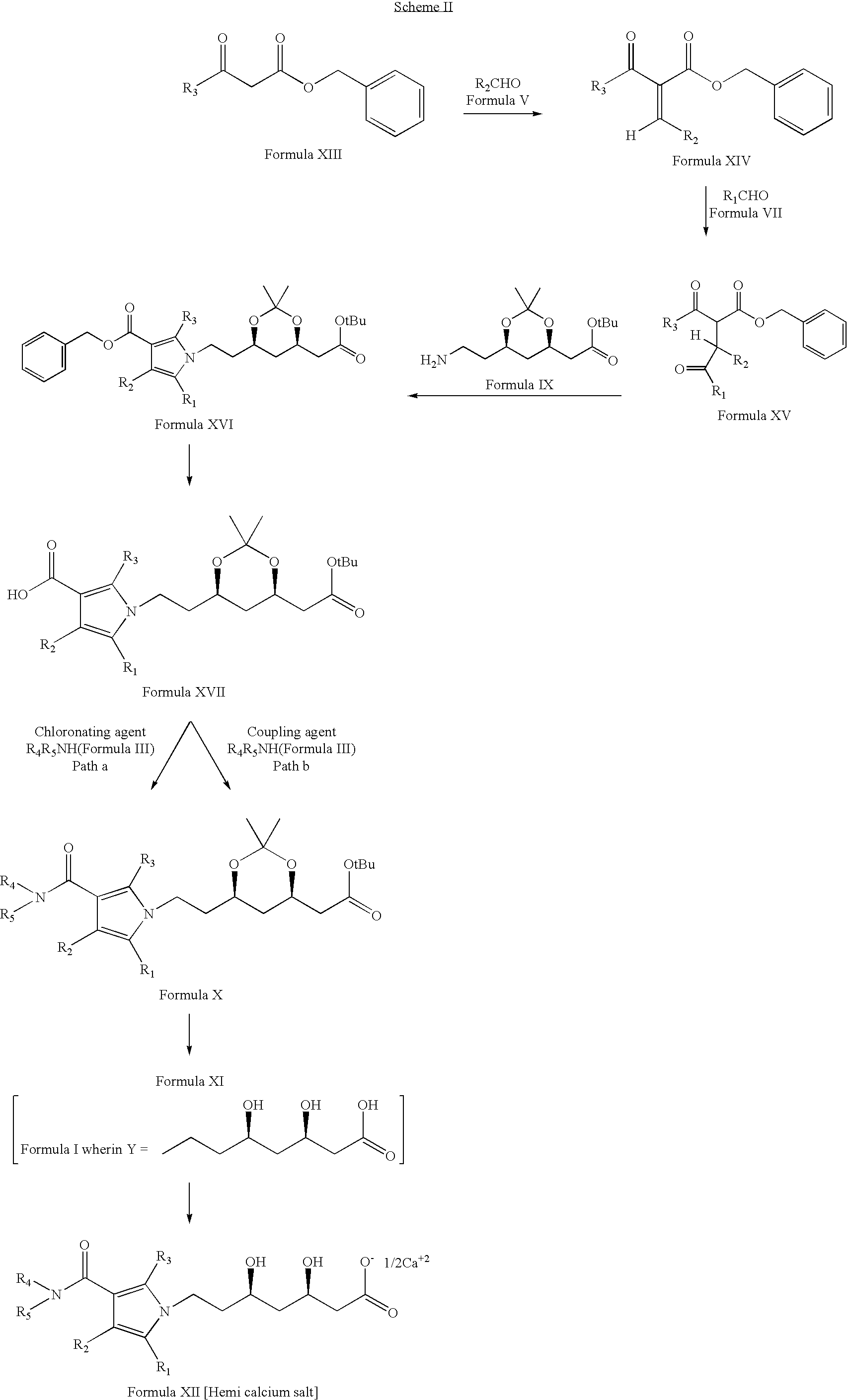

Compounds of Formula XII can also be prepared according to Scheme II. Accordingly, a compound of Formula XIII is reacted with a compound of Formula V to give a compound of Formula XIV (wherein R₂ and R₃ are as defined earlier in Scheme I) which, on reaction with a compound of Formula VII (wherein R₁ is as defined earlier), gives a compound of Formula XV, which on treatment with a compound of Formula IX yields a compound of Formula XVI, which on debenzylation gives a compound of Formula XVII, which on

a) conversion to corresponding acid chloride followed by reaction with an amine of Formula III (Path a) or

b) reaction with an amine of Formula III in the presence of a coupling agent (Path b) gives a compound of Formula X, which on hydrolysis gives a compound of Formula XI, which can be further converted to hemicalcium salt of Formula XI by following procedures well-known in the art.

The reaction of a compound of Formula XIII with an aldehyde of Formula V to give a compound of Formula XIV can be carried out in a nonpolar solvent, such as xylene, toluene, heptane, hexane or dichloromethane. The reaction of a compound of Formula XIII with a compound of Formula V can be carried out in the presence of an organic base, such as triethylamine, pyridine, piperidine or β-alanine and an organic acid such as glacial acetic acid or benzoic acid.

The reaction of a compound of Formula XIV with an aldehyde of Formula VII to give a compound of Formula XV can be carried out in a polar solvent, such as an alcoholic solvent, for example, methanol, ethanol, propanol or isopropanol. The reaction of a compound of Formula XIV with an aldehyde of Formula VII can be carried out in the presence of an organic base such as triethylamine or pyridine.

The reaction of a compound of Formula XIV with an aldehyde of Formula VII to give a compound of Formula XV can be carried out in the presence of a suitable catalyst such as sodium cyanide, 3-ethyl-5-(2-hydroxyethyl)-4-methyl thiazolium bromide or 3-benzyl-5-(2-hydroxyethyl)-4-methyl thiazolium chloride.

The reaction of a compound of Formula XV with an amine of Formula IX to give a compound of Formula XVI can be carried out in the presence of an acid, such as pivalic acid and p-toluene sulfonic acid in a nonpolar solvent such as hexane, heptane, toluene, tetrahydrofuran or a mixture thereof in a suitable ratio.

The debenzylation of a compound of Formula XVI to give a compound of Formula XVII can be carried out in the presence of a catalyst, such as palladium on carbon and hydrogen, in a polar solvent, such as methanol, ethanol, propanol or dioxane.

The conversion of compound of Formula XVII to its corresponding acid chloride (Path a) can be carried out with any suitable chlorinating agent, such as oxalyl chloride, in a nonpolar solvent, such as benzene, dichloromethane, tetrahydrofuran, toluene or xylene, followed by reaction with an amine of Formula III to give a compound of Formula X, in a nonpolar solvent, such as benzene, and in the presence of an organic base, such as triethylamine or pyridine.

Reaction of compound of Formula XVII with an amine of Formula III to give a compound of Formula X can be carried out in the presence of a coupling agent, such as O-benzotriazol-1-yl-N,N,N′,N′-tetramethyl uronium hexafluorophosphate (HBTU), bis(2-oxo-3-oxazolidinyl)phosphine (BOP), 1,3-dicyclohexycarbodiimide (DCC), 2-(1H-benzotriazole-1-yl)-1,1,3,3-tetramethyluronium tetrafluoroborate (TBTU), benzotriazole-1-yl-oxy-tris-pyrrolidino-phosphonium hexafluorophosphate (PyBOP) or carbonyldiimidazole (CDI) (Path b) in a polar solvent, such as dimethylformamide, and an organic base, such as diisopropylethylamine.

The conversion of a compound of Formula X to a compound of Formula XI can be carried out in a two-step manner, involving an initial acid-catalysed cleavage of ketal, followed by base-catalysed hydrolysis of the tert-butyl ester. The acid can be a mineral acid, such as hydrochloric acid. The cleavage of ketal can be carried out by any other cleavage method known in the prior art. The base can be an inorganic base, for example, lithium hydroxide, sodium hydroxide or potassium hydroxide.

The compound of Formula XI can be converted into its corresponding hemi calcium salt by following procedures well known to a person ordinary skilled in the art. The hemi calcium salts of compound of Formula XI can also be prepared from the corresponding lactones form of Formula XI by following procedures well known in the art.

An illustrative list of particular compounds disclosed herein is given below (also shown in Tables 1 and 2):
(3R,5R)-7-[2-(4-Fluorophenyl)-5-isopropyl-3-phenyl-4-(4-methylthiazol-2-ylamino)carbonyl]-pyrrol-1-yl]-3,5-dihydroxy-heptanoic acid (Compound No. 1)
(3R,5R)-7-[2-(4-Fluorophenyl)-5-isopropyl-3-phenyl-4-(benzothiazol-2-ylamino)carbonyl]-pyrrol-1-yl]-3,5-dihydroxy-heptanoic acid (Compound No. 2)
(3R,5R)-7-[2-(4-Fluorophenyl)-5-isopropyl-3-(pyridin-2-yl)-4-(phenylamino)carbonyl]-pyrrol-1-yl]-3,5-dihydroxy-heptanoic acid (Compound No. 3)
(3R,5R)-7-[2-(4-Fluorophenyl)-5-isopropyl-3-(pyridin-3-yl)-4-(phenylamino)carbonyl]-pyrrol-1-yl]-3,5-dihydroxy-heptanoic acid (Compound No. 4)
(3R,5R)-7-[2-(4-Fluorophenyl)-5-isopropyl-3-(pyridin-4-yl)-4-(phenylamino)carbonyl]-pyrrol-1-yl]-3,5-dihydroxy-heptanoic acid (Compound No. 5)
(3R,5R)-7-[2-(4-Fluorophenyl)-5-isopropyl-3-(5-methylfuran-2-yl)-4-(phenylamino)carbonyl]-pyrrol-1-yl]-3,5-dihydroxy-heptanoic acid (Compound No. 6)
(3R,5R)-7-[2-(4-Fluorophenyl)-5-isopropyl-3-(thiophen-2-yl)-4-(phenylamino)carbonyl]-pyrrol-1-yl]-3,5-dihydroxy-heptanoic acid (Compound No. 7)
(3R,5R)-7-[2-(4-Fluorophenyl)-5-isopropyl-3-(thiophen-3-yl)-4-(phenylamino)carbonyl]-pyrrol-1-yl]-3,5-dihydroxy-heptanoic acid (Compound No. 8)
(3R,5R)-7-[2-(4-Fluorophenyl)-5-isopropyl-3-phenyl-4-(1H-indol-5-yl-amino)carbonyl]-pyrrol-1-yl]-3,5-dihydroxy-heptanoic acid (Compound No. 9)
(3R,5R)-7-[2-(4-Fluorophenyl)-5-isopropyl-3-phenyl-4-(1-methyl-1H-indol-5-yl-amino)carbonyl]-pyrrol-1-yl]-3,5-dihydroxy-heptanoic acid (Compound No. 10)
(3R,5R)-7-[2-(4-fluorophenyl)-5-isopropyl-3-(pyridin-3-yl)-4-(4-acetylphenyl amino)carbonyl]-pyrrol-1-yl]-3,5-dihydroxy-heptanoic acid (Compound No. 11),
(3R,5R)-7-[2-(4-fluorophenyl)-5-isopropyl-3-(thiophen-2-yl)-4-(3-fluorophenyl amino)carbonyl]-pyrrol-1-yl]-3,5-dihydroxy-heptanoic acid (Compound No. 12),
(3R,5R)-7-[2-(4-fluorophenyl)-5-isopropyl-3-(thiophen-3-yl)-4-(3-fluorophenyl amino)carbonyl]-pyrrol-1-yl]-3,5-dihydroxy-heptanoic acid (Compound No. 13),
(3R,5R)-7-[2-(4-fluorophenyl)-5-isopropyl-3-(pyridin-4-yl)-4-(2,4-dimethoxyphenyl amino)carbonyl]-pyrrol-1-yl]-3,5-dihydroxy-heptanoic acid (Compound No. 14),
(3R,5R)-7-[2-(4-fluorophenyl)-5-isopropyl-3-(pyridin-3-yl)-4-(2,4-dimethoxyphenyl amino)carbonyl]-pyrrol-1-yl]-3,5-dihydroxy-heptanoic acid (Compound No. 15),
(3R,5R)-7-[2-(4-fluorophenyl)-5-isopropyl-3-(pyridin-4-yl)-4-(3-fluorophenyl amino)carbonyl]-pyrrol-1-yl]-3,5-dihydroxy-heptanoic acid (Compound No. 16),
(3R,5R)-7-[2-(4-fluorophenyl)-5-isopropyl-3-(pyridin-3-yl)-4-(4-methoxyphenyl amino)carbonyl]-pyrrol-1-yl]-3,5-dihydroxy-heptanoic acid (Compound No. 17),
(3R,5R)-7-[2-(4-fluorophenyl)-5-isopropyl-3-pyridin-3-yl)-4-(3-fluorophenyl amino)carbonyl]-pyrrol-1-yl]-3,5-dihydroxy-heptanoic acid (Compound No. 18),
(3R,5R)-7-[2-(4-fluorophenyl)-5-isopropyl-3-(pyridin-3-yl)-4-(2-hydroxyphenyl amino)carbonyl]-pyrrol-1-yl]-3,5-dihydroxy-heptanoic acid (Compound No. 19),
(3R,5R)-7-[2-(4-fluorophenyl)-5-isopropyl-3-(pyridin-3-yl)-4-(2-methoxyphenyl amino)carbonyl]-pyrrol-1-yl]-3,5-dihydroxy-heptanoic acid (Compound No. 20),
(3R,5R)-7-[2-(4-fluorophenyl)-5-isopropyl-3-(pyridin-4-yl)-4-(4-methoxyphenyl amino)carbonyl]-pyrrol-1-yl]-3,5-dihydroxy-heptanoic acid (Compound No. 21),
(3R,5R)-7-[2-(4-fluorophenyl)-5-isopropyl-3-(pyridin-4-yl)-4-(2-hydroxyphenyl amino)carbonyl]-pyrrol-1-yl]-3,5-dihydroxy-heptanoic acid (Compound No. 22),
(3R,5R)-7-[2-(4-fluorophenyl)-5-isopropyl-3-(pyridin-4-yl)-4-(2-methoxyphenyl amino)carbonyl]-pyrrol-1-yl]-3,5-dihydroxy-heptanoic acid (Compound No. 23),
(3R,5R)-7-[2-(3,4-difluorophenyl)-5-isopropyl-3-(thiophen-3-yl)-4-(phenyl amino)carbonyl]-pyrrol-1-yl]-3,5-dihydroxy-heptanoic acid (Compound No. 24),

and their lactone forms, pharmaceutically acceptable salts, pharmaceutically acceptable solvates, tautomers, racemates, polymorphs, pure enantiomers, diastereoisomers, metabolites, prodrugs or N-oxides.

TABLE 1

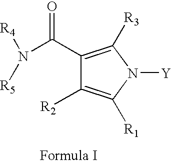

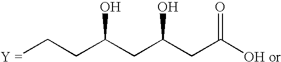

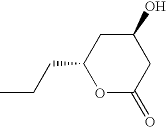

| C.No. | R₁ | R₂ | R₃ | R₄ | R₅ |
|---|---|---|---|---|---|
| 1 | 4-Fluorophenyl | Phenyl | Isopropyl | Hydrogen | 4-Methylthiazol-2-yl |
| 2 | 4-Fluorophenyl | Phenyl | Isopropyl | Hydrogen | Benzothiazol-2-yl |
| 3 | 4-Fluorophenyl | 2-Pyridyl | Isopropyl | Hydrogen | Phenyl |
| 4 | 4-Fluorophenyl | 3-Pyridyl | Isopropyl | Hydrogen | Phenyl |
| 5 | 4-Fluorophenyl | 4-Pyridyl | Isopropyl | Hydrogen | Phenyl |
| 6 | 4-Fluorophenyl | 5-Methyl-2-furyl | Isopropyl | Hydrogen | Phenyl |
| 7 | 4-Fluorophenyl | 2-Thienyl | Isopropyl | Hydrogen | Phenyl |
| 8 | 4-Fluorophenyl | 3-Thienyl | Isopropyl | Hydrogen | Phenyl |
| 9 | 4-Fluorophenyl | Phenyl | Isopropyl | Hydrogen | Indolin-5-yl |
| 10 | 4-Fluorophenyl | Phenyl | Isopropyl | Hydrogen | 1-Methylindolin-5-yl |

TABLE 2

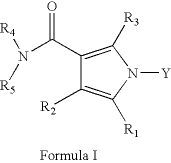

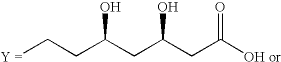

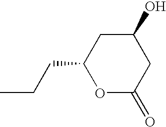

| C. No. | R₁ | R₂ | R₃ | R₄ | R₅ |
|---|---|---|---|---|---|
| 11 | 4-Fluorophenyl | Pyridin-3-yl | Isopropyl | Hydrogen | 4-Acetylphenyl |
| 12 | 4-Fluorophenyl | Thiophen-2-yl | Isopropyl | Hydrogen | 3-Fluorophenyl |
| 13 | 4-Fluorophenyl | Thiophen-3-yl | Isopropyl | Hydrogen | 3-Fluorophenyl |
| 14 | 4-Fluorophenyl | Pyridin-4-yl | Isopropyl | Hydrogen | 2,4-Dimethoxyphenyl |
| 15 | 4-Fluorophenyl | Pyridin-3-yl | Isopropyl | Hydrogen | 2,4-Dimethoxyphenyl |
| 16 | 4-Fluorophenyl | Pyridin-4-yl | Isopropyl | Hydrogen | 3-Fluorophenyl |
| 17 | 4-Fluorophenyl | Pyridin-3-yl | Isopropyl | Hydrogen | 4-Methoxyphenyl |
| 18 | 4-Fluorophenyl | Pyridin-3-yl | Isopropyl | Hydrogen | 3-Fluorophenyl |
| 19 | 4-Fluorophenyl | Pyridin-3-yl | Isopropyl | Hydrogen | 2-Hydroxyphenyl |
| 20 | 4-Fluorophenyl | Pyridin-3-yl | Isopropyl | Hydrogen | 2-Methoxyphenyl |
| 21 | 4-Fluorophenyl | Pyridin-4-yl | Isopropyl | Hydrogen | 4-Methoxyphenyl |
| 22 | 4-Fluorophenyl | Pyridin-4-yl | Isopropyl | Hydrogen | 2-Hydroxyphenyl |
| 23 | 4-Fluorophenyl | Pyridin-4-yl | Isopropyl | Hydrogen | 2-Methoxyphenyl |
| 24 | 3,4-difluorophenyl | Thiophen-3-yl | Isopropyl | Hydrogen | Phenyl |

The term “pharmaceutically acceptable” means approved by regulatory agency of the federal or a state government or listed in the U.S. Pharmacopoeia or other generally recognized pharmacopeia for use in animals, and more particularly in humans.

The term “pharmaceutically acceptable salts” refer to a salt prepared from pharmaceutically acceptable monovalent, divalent or trivalent non-toxic metal or organic base. Examples of such metal salts include, but are not limited to, lithium, sodium, potassium, calcium, magnesium, zinc, aluminum, and the like. Examples of such organic bases include, but are not limited to, amino acid, ammonia, mono-alkyl ammonium, dialkyl ammonium, trialkyl ammonium and N-methyl glucamine and the like. Preferably, this invention contemplates calcium salts of compounds as disclosed herein. The free acid forms of compounds of the present invention may be prepared from the salt forms, if desired, by contacting the salt with dilute aqueous solution of an acid, such as hydrochloric acid. The base addition salts may differ from the free acid forms of the compounds of this invention in such physical characteristics as solubility and melting point.

The term “pharmaceutically acceptable solvates” refers to solvates with water (i-e hydrates) or pharmaceutically acceptable solvents, for example solvates with ethanol and the like. Such solvates are also encompassed within the scope of the disclosure. Furthermore, some of the crystalline forms for compounds described herein may exist as polymorphs and as such are intended to be included in the scope of the disclosure.

The present invention also includes within its scope prodrugs of these agents. In general, such prodrugs will be functional derivatives of these compounds, which are readily convertible in vivo into the required compound. Conventional procedure for the selection and preparation of suitable prodrug derivatives are described, for example, in “design of prodrugs”, ed. H Bundgaard and, Elsevier, 1985.

The disclosed compounds may get metabolized in vivo and these metabolites are also encompassed within the scope of this invention.

The compounds of the invention possess two chiral centers, they may, therefore, exist as enantiomers and diastereomers. It is to be understood that all such isomers and racemic mixtures therefore are encompassed within the scope of the present invention. Preferably, this invention contemplates compounds only with 3R and 5R configuration.

The crystalline or amorphous forms of compounds disclosed herein may exist as polymorphs and as such are intended to be included in the present invention.

Pharmaceutical compositions comprising compounds disclosed herein, their pharmaceutically acceptable salt, pharmaceutically acceptable solvates, or polymorphs, and pharmaceutically acceptable carrier or excipient are also disclosed herein.

The compositions provided herein, both those containing one disclosed compound and those containing two or more of such compounds, may be suitable for oral or parenteral administration. The compositions may be formulated to provide immediate or sustained release of the therapeutic compounds. The compounds described herein can be administered alone but will generally be administered as an admixture with suitable pharmaceutically acceptable carriers. The term “pharmaceutically acceptable carrier” is intended to include non-toxic, inert solid, semi-solid, liquid filter, diluent, encapsulating materials or formulation auxiliaries of any type.

Solid form preparations for oral administration may include capsules, tablets, pills, powder, granules or suppositories. For solid form preparations, the active compound is mixed with at least one inert, pharmaceutically acceptable excipient or carrier such as sodium citrate, dicalcium phosphate and/or a filler, an extender, such as starch, lactose, sucrose, glucose, mannitol or silicic acid; binders, such as carboxymethyl cellulose, alginates, gelatins, polyvinylpyrrolidinone, sucrose, or acacia; disintegrating agents, such as agar-agar, calcium carbonate, potato starch, aliginic acid, certain silicates or sodium carbonate; absorption accelerators, such as quaternary ammonium compounds; wetting agents, such as cetyl alcohol, glycerol, or mono stearate adsorbents such as Kaolin; lubricants, such as talc, calcium stearate, magnesium stearate, solid polyethyleneglycol, or sodium lauryl sulphate, and mixtures thereof.

In case of capsules, tablets, and pills, the dosage form may also comprise buffering agents.

The solid preparation of tablets, capsules, pills, or granules can be accomplished with coatings and/or shells such as enteric coatings and other coatings well known in the pharmaceutical formulating art.

Liquid form preparations for oral administration can include pharmaceutically acceptable emulsions, solutions, suspensions, syrups and elixirs. For liquid form preparations, the active compound can be mixed with water or other solvent, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (such as cottonseed, ground corn, germ, live, caster and sesamine oil), glycerol and fatty acid ester of sorbitan and mixture thereof.

Besides inert diluents, the oral compositions can also include adjuvants such as wetting agents, emulsifying agents, suspending agents, sweetening agents, flavoring agents and perfuming agents.

The formulations as described herein may be formulated so as to provide quick, sustained, or delayed release of the active compound after administration to the patient by employing procedures well-known to the art. The term “patient” as used herein refers to a human or nonhuman mammal, which is the object of treatment, observation or experiment.

The pharmaceutical preparations can be in unit dosage forms, in such form, the preparations are subdivided into unit doses containing appropriate quantities of an active compound.

The amount of a compound disclosed herein that will be effective in the treatment of a particular disorder or condition can be determined by standard clinical techniques. In addition, in vitro or in vivo assays may optionally be employed to help identify optimal dosage ranges.

Examples set forth below demonstrate general synthetic procedures for preparation of particular representative compounds. The examples are provided to illustrate particular aspects of the disclosure, and do not constrain the scope of the present invention as defined by the claims.

### EXPERIMENTAL DETAILS

### General Procedure

Scheme I

Step 1: Preparation of β Ketoamide-1 (Formula IV)

A mixture of β ketoester (Formula II, 1 equiv) amine (Formula III, 1 equiv) 1,2-ethylene diamine (0.01 equiv) in xylene was refluxed with the azeotrpic removal of water. After the completion of reaction, solvent was evaporated & the residue purified on column (silica gel; 100-200 mesh).

The following intermediates were prepared following above general procedure.

4-Methyl-3-oxo-pentanoic acid (4-methylthiazol-2-yl) amide

¹H NMR (CDCl₃, 300 MHz): δ 1.16 (d, J=6 Hz, 6H), 2.35 (s, 3H), 2.73 (sept, J=6 Hz, 1H), 3.68 (s, 2H), 6.53 (s, 1H); MS (positive ion mode): m/z 227 (M⁺+1)

4-Methyl-3-oxo-pentanoic acid (4-acetylphenyl) amide

¹H NMR (CDCl₃, 300 MHz): δ 1.16 (d, J=6.9 Hz, 6H), 2.74 (sep. J=6.9 Hz), 3.64 (s, 2H), 3.9 (s, 3H), 7.64 (d, J=8.7 Hz, 2H), 8.00 (d, J=8.7 Hz, 2H), 9.56 (s, 1H); MS (Positive ion mode): m/z 248 (M⁺+1); Yield: 90%.

4-Methyl-3-oxo-pentanoic acid (3-fluorophenyl) amide

¹H NMR (CDCl₃): δ 1.15 (d, J=6.9 Hz, 6H), 2.73 (sep, J=6.9 Hz, 1H), 3.617 (s, 2H), 6.80 (t, J=7.2 Hz, 1H), 7.16-7.24 (m, 2H), 7.52 (d, J=11.1 Hz, 1H), 9.41 (bs, 1H); MS (Positive ion mode): m/z 224.3 (M⁺+1); Yield: 60.03%.

4-Methyl-3-oxo-pentanoic acid (2,4-dimethoxyphenyl) amide

¹H NMR (CDCl₃, 300 MHz): δ 1.18 (d, J=6 Hz, 6H), 2.73 (sep, J=6 Hz, 1H), 3.6 (s, 2H), 3.79 (s, 3H), 3.89 (s, 3H), 6.43-6.48 (m, 2H), 8.18 (d, J=9 Hz, 1H), 9.2 (brs, 1H); Yield: 61.59%.

4-Methyl-3-oxo-pentanoic acid (4-methoxyphenyl) amide

¹H NMR (CDCl₃, 300 MHz): δ 1.16 (d, 3H), 1.18 (d, 3H), 2.72-2.76 (m, 1H), 3.59 (s, 2H), 3.79 (s, 3H), 6.88 (d, 2H, J=9 Hz), 7.45 (d, J=9 Hz, 2H), 9.08 (brs, —NH); MS (Positive ion mode): m/z 236 (M⁺+1); Yield: 98.7%.

4-Methyl-3-oxo-pentanoic acid (2-methoxyphenyl) amide

¹H NMR (CDCl₃, 300 MHz): δ 1.17 (d, J=6 Hz, 6H), 2.76 (m, 1H), 3.62 (s, 2H), 3.93 (s, 3H), 6.87-7.08 (m, 3H), 8.33 (d, J=9 Hz, 1H), 9.39 (S, 1H); MS (Positive ion mode): m/z 236; M⁺+1); Yield: 86%.

4-Methyl-3-oxo-pentanoic acid (2-benzyloxyphenyl) amide

¹H NMR (CDCl₃, 300 MHz): δ 1.15 (d, J=8.8 Hz, 6H), 2.72 (sep, J=6.9 Hz, 1H), 3.59 (s, 2H), 5.17 (s, 2H), 6.93-7.03 (m, 3H), 7.33-7.42 (m, 3H), 7.50-7.54 (m, 2H), 8.34 (d, J=6 Hz, 1H), 9.5 (brs, 1H); MS (Positive ion mode): m/z 312.40 (M⁺+1); Yield: 79.5%.

4-Methyl-3-oxo-pentanoic acid phenylamide

Step 2: Preparation of β-ketoamide-2 (Formula VI)

β-ketoamide-1 (Formula IV, 1 equiv) in hexane was added β-alanine (0.18 equiv), aldehyde (Formula V, 1.1 equiv) and glacial acetic acid (0.16% w/w of β-ketoamide-1). The resulting suspension was heated under reflux with the azeotropic removal of water. The reaction mixture was cooled and product was isolated by filtration. The product was purified by washing the precipitate with hot hexane, water and dried in vacuo to afford β-ketoamide-2.

The following intermediates were prepared following above general procedure.

2-Benzylidene-4-methyl-3-oxo-pentanoic acid (4-methyl-thiazol-2-yl) amide

4-Methyl-3-oxo-2-(pyridin-2-yl)-methylene-pentanoic acid phenylamide

¹H NMR (CDCl₃, 300 MHz): δ 1.17 (d, J=6 Hz, 6H), 2.84 (sept, J=6 Hz, 1H), 7.11-7.96 (m, 8H), 8.59 (d, J=6 Hz, 1H), 8.75 (s, 1H); MS (positive ion mode): m/z 295 (M⁺+1); Yield: 28%.

4-Methyl-3-oxo-2-(pyridin-3-yl)-methylene-pentanoic acid phenylamide

¹H NMR (CDCl₃): δ 1.24 (d, J=6.9 Hz, 6H), 3.38 (sep, J=6.6 Hz, 1H), 7.15 (t, J=7.5 Hz, 1H), 7.18-7.40 (m, 3H), 7.55 (m, 3H), 7.98 (d, J=9 Hz, 1H), 8.18 (s, 1H), 8.56 (d, J=3.9 Hz, 1H), 8.62 (s, 1H); MS (positive ion mode): m/z 295 (M⁺+1); Yield: 40%.

4-Methyl-3-oxo-2-(pyridin-4-yl)-methylene-pentanoic acid phenylamide

¹H NMR (DMSO-d₆, 300 MHz): δ 1.12 (d, J=6 Hz, 6H), 3.40 (Sept, J=6 Hz, 1H), 7.11 (t, J=6 Hz, 1H), 7.34 (t, J=6 Hz, 2H), 7.53-7.60 (m, 4H), 7.71 (s, 1H), 8.62 (d, J=6 Hz, 1H), 10.52 (s, 1H); MS (positive ion mode): m/z 295 (M⁺+1); Yield: 42%.

4-Methyl-2-(5-methyl-furan-2-yl)-methylene-3-oxo-pentanoic acid phenylamide

¹H NMR (CDCl₃, 300 MHz): δ 1.19 (d, J=6.6 Hz, 6H), 2.22 (s, 3H), 3.32 (sept, J=6.6 Hz, 1H), 6.13 (d, J=1.8 Hz, 1H), 7.03 (d, J=3.3 Hz, 1H), 7.15 (t, J=7.2 Hz, 1H), 7.37 (t, J=7.8 Hz, 2H), 7.43 (s, 1H), 7.62 (d, J=8.1 Hz, 2H), 8.14 (s, 1H); MS (positive ion mode): m/z 300 (M⁺+1); Yield: 82%.

4-methyl-3-oxo-2-(thiophen-2-yl)-methylene-pentanoic acid phenylamide

¹H NMR (CDCl₃, 300 MHz): δ 1.22 (d, J=6 Hz, 6H), 3.38 (sept, J=6 Hz, 1H), 7.09-7.19 (m, 2H), 7.38 (t, J=9 Hz, 2H), 7.49 (d, J=3 Hz, 1H), 7.59 (d, J=3 Hz, 1H), 7.66 (d, J=9 Hz, 2H), 7.86 (s, 1H), 8.70 (brs, 1H); MS (positive ion mode): m/z 299 (M⁺+1).

4-methyl-3-oxo-2-(thiophen-3-yl)-methylene-pentanoic acid phenylamide

¹H NMR (CDCl₃): δ 1.21 (d, J=6 Hz, 6H), 3.32 (sept, J=6.0 Hz, 1H), 7.17 (t, J=6 Hz, 1H), 7.25-7.42 (m, 4H), 7.59 (d, J=12 Hz, 3H), 7.75 (s, 1H), 7.84 (s, 1H); MS (positive ion mode): m/z 300 [M+1]; Yield: 70%.

4-Methyl-3-oxo-2-(pyridin-3-yl)-methylene-pentanoic acid (4-acetylphenyl) amide

¹H NMR (300 MHz): δ 1.24 (d, J=6.9 Hz, 6H), 2.59 (s, 3H), 3.36 (sep, J=6.6 Hz, 1H), 7.23-7.33 (m, 1H), 7.52 (s, 1H), 7.69 (d, J=8.7 Hz, 2H), 7.90-8.02 (m, 3H), 8.51-8.63 (m, 2H), 8.84 (s, 1H); MS (Positive ion mode): m/z 337.7 (M⁺+1); Yield: 53.66%.

4-Methyl-3-oxo-2-(thiophen-2-yl)-methylene-pentanoic acid (3-fluorophenyl) amide

¹H NMR (CDCl₃): δ 1.21 (d, J=6 Hz, 6H), 3.36 (sep, J=6 Hz, 1H), 6.82-6.87 (m, 1H), 7.09 (t, J=6 Hz, 1H), 7.28-7.3 (m, 2H), 7.46 (d, J=3 Hz, 1H), 7.60-7.67 (m, 2H), 7.84 (s, 1H), 9.14 (bs, 1H); MS (Positive ion mode): m/z 318.4 (M⁺+1); Yield: 86.5%.

4-Methyl-3-oxo-2-(thiophen-3-yl)-methylene-pentanoic acid (3-fluorophenyl) amide

¹H NMR (CDCl₃, 300 MHz): δ 1.22 (d, J=6 Hz, 6H), 3.3-3.34 (m, 1H), 6.84-6.9 (m, 1H), 7.21-7.32 (m, 5H), 7.61 (brs, 2), 7.77 (brs, 1H), 8.04 (brs, 1H); MS Positive ion mode): m/z 318; (M⁺+1); Yield: 62.37%.

4-Methyl-3-oxo-2-(pyridin-4-yl)-methylene-pentanoic acid (2,4-dimethoxy phenyl) amide

¹H NMR (300 MHz): δ 1.05 (d, J=6 Hz, 3H), 1.21 (d, J=9 Hz, 6H), 2.52 (sep, J=6 Hz, 0.6H), 3.69 (s, 3H), 3.81 (s, 4.7H), 3.92 (s, 1.3H), 6.43-6.53 (m, 2.9H), 7.20 (d, J=6.0 Hz, 0.6H), 7.40 (d, J=6.0 Hz, 2H), 7.50 (s, 1H), 7.91 (d, J=9.0 Hz, 1H), 8.21-8.30 (m, 1.4H), 8.60 (d, J=6.0 Hz, 2H), 8.67 (d, J=6.0 Hz, 0.9H)

4-Methyl-3-oxo-2-(pyridin-3-yl)-methylene-pentanoic acid (2,4-dimethoxyphenyl) amide Isomer (1)

¹H NMR (CDCl₃, 300 MHz): δ 1.22 (d, J=6 Hz, 6H), 3.34 (sep, J=6 Hz, 1H), 3.71 (s, 3H), 3.81 (s, 3H), 6.44 (s, 1H), 6.5-6.53 (m, 1H), 7.58 (s, 1H), 7.93 (d, J=9 Hz, 1H), 7.99-8.01 (m, 1H), 8.26 (d, J=9 Hz, 1H), 8.56 (d, J=3 Hz, 1H), 8.64 (s, 1H); MS (Positive ion mode): m/z 355.19 (M⁺+1); Yield: 41.8%;

4-Methyl-3-oxo-2-(pyridin-3-yl)-methylene-pentanoic acid (2,4 -dimethoxyphenyl) amide Isomer (2)

¹H NMR (CDCl₃, 300 MHz): δ 1.07 (d, J=6 Hz, 6H), 2.59 (sep. J=6 Hz, 1H), 3.81 (s, 3H), 3.92 (s, 3H), 6.50 (d, J=3 Hz, 2H), 7.33-7.37 (m, 1H), 7.63 (d, J=9 Hz, 1H), 8.01 (s, 1H), 8.3 (d, J=3 Hz, 1H), 8.59 (s, 1H), 8.63 (d, J=6 Hz, 1H), 9.14 (s, 1H); MS (Positive ion mode): m/z 355.19 (M⁺+1); Yield: 24.22%.

4-Methyl-3-oxo-2-(pyridin-4-yl)-methylene-pentanoic acid (3-fluorophenyl) amide

¹H NMR (CDCl₃, 300 MHz): δ 1.2 (d, J=6.9 Hz, 6H), 3.3 (sep. J=6.9 Hz, 1H), 6.86 (dd, J=8.4 & 8.1 Hz, 1H), 7.13 (d, J=8.1 Hz, 1H), 7.23-7.34 (m, 3H), 7.43 (s, 1H), 7.49 (d, J=10.2 Hz, 1H), 8.54 (d, J=4.8 Hz, 2H), 8.71 (s, 1H); MS (Positive ion mode): m/z 313.5 (M⁺+1); Yield: 69.52%

4-Methyl-3-oxo-2-(pyridin-3-yl)-methylene-pentanoic acid (4-methoxy phenyl) amide

¹H NMR (CDCl₃, 300 MHz): δ 1.26 (d, 3H), 1.24 (d, 3H), 3.30-3.37 (m, 1H), 3.81 (s, 3H), 6.87-6.90 (d, 2H, J=9 Hz), 7.26-7.29 (d, 2H, J=9 Hz), 7.43-7.46 (d, 2H, J=9 Hz), 7.51 (s, 1H), 7.95-7.98 (d, 2H, J=9 Hz), 8.21 (brs, 1H, —NH), 8.54-8.56 (d, 2H, J=6 Hz); MS (Positive ion mode): m/z 325 (M⁺+1); Yield: 72.79%.

4-Methyl-3-oxo-2-(pyridin-3-yl)-methylene-pentanoic acid (3-fluorophenyl) amide

¹H NMR (CDCl₃, 300 MHz): δ 1.25 (d, J=6 Hz, 6H), 3.3 (sep. J=6 Hz, 1H), 6.86 (dd, J=9 & 6 Hz, 1H), 7.2-7.32 (m, 3H), 7.41 (s, 1H),7.58 (d, J=12 Hz, 1H), 7.95 (d, J=9 Hz, 1H), 8.26 (s, 1H), 8.48 (d, J=3 Hz, 1H), 9.24 (s, 1H); MS (Positive ion mode): m/z 313.4 (M⁺+1); Yield: 65.43%.

4-Methyl-3-oxo-2-(pyridin-3-yl)-methylene-pentanoic acid (2-benzyloxyphenyl) amide

¹H NMR (CDCl₃, 300 MHz): δ 1.20 (d, J=6 Hz, 6H), 3.32 (sep, J=6 Hz, 1H), 4.99 (s, 2H), 6.92 (d, J=9 Hz, 1H), 7.0-7.15 (m, 2H), 7.16-7.18 (m, 2H), 7.31-7.33 (m, 5H), 7.56 (s, 1H), 7.9-8.0 (m, 1H), 8.25 (brs, 1H), 8.35-8.45 (m, 1H), 8.50-8.60 (m, 1H), 8.73 (brs, 1H).

4-Methyl-3-oxo-2-(pyridin-3-yl)-methylene-pentanoic acid (2-methoxy phenyl) amide

¹H NMR (CDCl₃, 300 MHz): δ 1.11 (d, J=6 Hz, 6H), 3.33 (sep, J=6 Hz, 1H), 3.74 (s, 3H), 6.85 (d, J=9 Hz, 1H), 7.0-7.15 (m, 2H), 7.20-7.26 (m, 1H), 7.59 (s, 1H), 7.90-8.05 (m, 1H), 8.18 (brs, 1H), 8.42 (d, J=6 Hz, 1H), 8.56 (d, J=6 Hz, 1H), 8.74 (brs, 1H); MS (Positive ion mode): m/z 325.38 (M⁺+1); Yield: 56%.

4-Methyl-3-oxo-2-(pyridin-4-yl)-methylene-pentanoic acid (4-methoxy phenyl) amide

¹H NMR (CDCl₃, 300 MHz): δ 1.25 (d, J=6 Hz, 6H), 3.34 (sep, J=6H, 1H), 3.80 (s, 3H), 6.88 (d, J=6 Hz, 2H), 7.36-7.39 (m, 4H), 7.47 (s, 1H), 7.84 (brs, 1H), 8.6 (brs, 1H); MS (Positive ion mode): m/z 325.37 (M⁺+1); Yield: 53%.

4-Methyl-3-oxo-2-(pyridin-4-yl)-methylene-pentanoic acid (2-benzyloxyphenyl) amide

¹H NMR (300 MHz): δ 0.96 (d, J=6.9 Hz, 6H), 2.47 (sep, J=6 Hz, 1H), 5.18 (s, 2H), 5.30 (s, 2H), 6.85-7.15 (m, 3H), 7.18 (d, J=6 Hz, 2H), 7.32-7.54 (m, 5H), 7.94 (s, 1H), 8.49 (d, J=6 Hz, 1H), 8.66 (d, J=6 Hz, 2H), 9.23 (brs, 1H); MS (Positive ion mode): m/z 401.43 [M⁺+1]; Yield: 79.3%.

4-Methyl-3-oxo-2-(pyridin-4-yl)-methylene-pentanoic acid (2-methoxy phenyl) amide

¹H NMR (CDCl₃, 300 MHz): δ 1.22 (d, J=6.9 Hz, 6H), 3.22 (sep, J=6.9 Hz, 1H), 3.73 (s, 3H), 6.85 (d, J=9 Hz, 1H), 7.0-7.15 (m, 2H), 7.43 (d, J=6 Hz, 2H), 7.51 (s, 1H), 8.12 (brs, 1H), 8.38 (d, J=7.8 Hz, 1H), 8.61 (d, J=6 Hz, 2H); MS (Positive ion mode): m/z 325.31 (M⁺+1); Yield: 22.2%.

Step 3: Preparation of Diketone (Formula VIII)

β-ketoamide-2 (Formula VI, 1 equiv), aldehyde (Formula VII, 1.1 equiv), triethylamine (1 equiv) ethanol and 3-ethyl-5-(2-hydroxyethyl)-4-methyl thiazolium bromide (0.2 equiv) were placed in a vial. The contents were flushed with N₂ and the vial capped immediately and heated to 78° C. After the completion of reaction, contents were cooled and triturated with ethyl acetate. The organic layer was washed with 6N hydrochloric acid, water, dried over anhydrous sodium sulphate, concentrated by rotary evaporation and residue purified on a chromatographic column (silica gel, 100-200 mesh)

The following intermediates were prepared following above general procedure:

2-[2-(4-Fluorophenyl)-2-oxo-1-phenyl-ethyl]-4-methyl-3-oxo-pentanoic acid (5-methylthiazol-2-yl) amide

MS (positive ion mode): m/z 438 (M⁺+1).

2-[2-(4-Fluoropheayl)-2-oxo-1-pyridin-2-yl-ethyl]-4-methyl-3-oxo-pentanoic acid phenylamide

¹H NMR (CDCl₃, 300 MHz): δ 1.16 (d, J=6 Hz, 3H), 1.24 (d, J=6 Hz, 3H), 3.06 (sept, J=6 Hz, 1H), 4.94 (d, J=12 Hz, 1H), 5.60 (d, J=12 Hz, 1H), 7.03-7.08 (m, 4H), 7.22-7.25 (m, 3H), 7.33 (d, J=9 Hz, 1H), 7.56 (t, J=9 Hz, 1H), 7.76 (s, 1H), 8.01-8.06 (m, 2H), 8.49 (d, J=6 Hz, 1H); MS (positive ion mode): m/z 419 (M⁺+1); Yield: 9%.

2-[2-(4-Fluorophenyl)-2-oxo-1-pyridin-3-yl-ethyl]-4-methyl-3-oxo-pentanoic acid phenylamide

¹H NMR (CDCl₃): δ 1.09 (d, J=6.6 Hz, 3H), 1.25 (d, J=6.6 Hz, 3H), 3.06 (sept, J=6.8 Hz, 1H), 5.32 (d, J=10.7, 1H), 5.63 (d, J=10.8, 1H), 6.93-7.33 (m, 5H), 7.45 (d, J=7.6, 3H), 8.02-8.14 (m, 3H), 8.47 (d, J=4.7 Hz, 1H), 9.08 (s, 1H), 9.79 (s, 1H); MS (positive ion mode): m/z 419 (M⁺+1); Yield: 46%.

2-[2-(4-Fluorophenyl)-2-oxo-1-pyridin-4-yl-ethyl]-4-methyl-3-oxo-pentanoic acid phenylamide

¹H NMR (CDCl₃, 300 MHz): δ 1.08 (d, J=6.6 Hz, 3H), 1.15 (d, J=6.6 Hz, 3H), 2.98 (sept, J=6.6 Hz, 1H), 4.51 (d, J=10.5 Hz, 1H), 5.38 (d, J=10.5 Hz, 1H), 7.05-7.32 (m, 9H), 7.94-7.99 (m, 2H), 8.50 (d, J=4.8 Hz, 2H); MS (positive ion mode): m/z 419 (M⁺+1); Yield: 18%.

2-[2-(4-Fluorophenyl)-1-(5-methylfuran-2-yl)-2-oxo-ethyl]-4-methyl-3-oxo-pentanoic acid phenylamide

¹H NMR (CDCl₃, 300 MHz): (3:1 mixture of diastereomer) δ 0.99 (d, J=6.9 Hz, 1H), 1.04 (d, J=6.9 Hz, 1H), 1.15 (d, J=6.9 Hz, 3H), 1.24 (d, J=6.9 Hz, 3H), 2.13 (s, 3H), 2.17 (s, 1H), 2.80 (Sept, J=6.9 Hz, 0.3H), 2.97 (Sept, J=6.9 Hz, 1H), 4.66 (d, J=11 Hz, 1.3H), 5.46 (d, J=11 Hz, 1H), 5.85 (d, J=11 Hz, 0.3H), 5.83 (brs, 1.3H), 6.07 (d, J=3 Hz, 0.3H), 6.1 (d, J=3 Hz, 1H), 7.05-7.14 (m, 4.5H), 7.29-7.45 (m, 7.2H); MS (positive ion mode): m/z 422 (M⁺+1); Yield: 56%.

2-[2-(4-Fluorophenyl)-2-oxo-1-thiophen-2-yl-ethyl]-4-methyl-3-oxo-pentanoic acid phenylamide

¹H NMR (CDCl₃, 300 MHz): δ 1.14 (d, J=6.9 Hz, 3H), 1.21 (d, J=7.2 Hz, 3H), 2.94 (sept, J=6.9 Hz, 1H), 4.57 (d, J=10.5 Hz, 1H), 5.66 (d, J=10.8 Hz, 1H), 6.87-6.96 (m, 3H), 7.05-7.11 (m, 4H), 7.26-7.31 (m, 3H), 8.01-8.06 (m, 2H).

2-[2-(4-Fluorophenyl)-2-oxo-1-thiophen-3-yl-ethyl]-4-methyl-3-oxo-pentanoic acid phenylamide

¹H NMR (CDCl₃): δ 1.14 (d, J=6 Hz, 3H), 1.21 (d, J=6 Hz, 3H), 2.94 (sept, J=6 Hz, 1H), 4.52 (d, J=9 Hz, 1H), 5.53 (d, J=9 Hz, 1H), 6.96-7.37 (m, 10H), 7.42-7.41 (d, J=6 Hz, 1H), 7.92-8.12 (m, 2H); MS (positive ion mode): m/z 424 [M+1]; Yield: 77%.

2-[2-(4-Fluorophenyl)-2-oxo-1-pyridin3-yl-ethyl]-4-methyl-3-oxo-pentanoic acid (4-acetylphenyl) amide

¹H NMR (300 MHz): δ 1.12 (d, J=6 Hz, 3H), 1.2 (d, J=6 Hz, 3H), 2.54 (s, 3H), 2.99 (sep, J=6 Hz, 1H), 4.77 (d, J=12 Hz, 1H), 5.50 (d, J=9 Hz, 1H), 7.09 (t, J=6 Hz, 2H), 7.25-7.40 (m, 3H), 7.78 (t, J=6 Hz, 3H), 7.96 (t, J=6 Hz, 2H), 8.38 (s, 1H), 8.52 (d, J=3 Hz, 1H), 9.27 (s, 1H); MS (Positive ion mode): m/z 461.5; Yield: 48%.

2-[2-(4-Fluorophenyl)-2-oxo-1-thiophen-2-yl-ethyl]-4-methyl-3-oxo-pentanoic acid (3-fluorophenyl) amide

¹H NMR (CDCl₃, 300 MHz): δ 0.87 (d, J=6.9 Hz, 3H), 0.99 (d, J=6.9 Hz, 3H), 1.14 (d, J=6.9 Hz, 3H), 1.18 (d, J=6.9 Hz, 3H), 2.94 (sep, J=6.9 Hz, 1H), 3.25 (m, 1H), 4.59 (d, J=10.5 Hz, 1H), 4.63 (m, 2H), 5.66 (d, J=10.5 Hz, 1H), 6.78-6.95 (m, 6H), 7.06-7.25 (m, 10H), 8.05 (t, J=8.7 Hz, 2H); MS (Positive ion mode): m/z 442.6 (M⁺+1) ; Yield: 51%.

2-[2-(4-Fluorophenyl)-2-oxo-1-thiophen-3-yl-ethyl]-4-methyl-3-oxo-pentanoic acid (3-fluorophenyl) amide

MS (Positive ion mode) m/z 442.5 (M⁺+1); Yield: 57.55%.

2-[2-(4-Fluorophenyl)-2-oxo-1-pyridin-4-yl-ethyl]-4-methyl-3-oxo-pentanoic acid (2,4-dimethoxyphenyl) amide

¹H NMR (CDCl₃, 300 MHz): δ 1.15 (d, J=7.8 Hz, 3H), 1.21 (d, J=9 Hz, 3H), 2.95 (sep, J=6.9 Hz, 1H), 3.76 (s, 6H), 4.52 (d, J=10.8 Hz, 1H), 5.37 (d, J=10.8 Hz, 1H), 6.40 (brs, 2H), 7.07 (t, J=9 Hz, 2H), 7.23-7.24 (m, 2H), 7.47 (s, 1H), 7.83 (d, J=9 Hz, 1H), 7.95-8 (m, 2H), 8.47 (d, J=5.1 Hz, 2H); MS (Positive ion mode): m/z 479.40 (M⁺+1); Yield: 24.77%.

2-[2-(4-Fluorophenyl)-2-oxo-1-pyridin-3-yl-ethyl]-4methyl-3-oxo-pentanoic acid (2,4 -dimethoxyphenyl) amide

¹H NMR (CDCl₃, 300 MHz): δ 1.13 (d, J=6 Hz, 3H), 1.18 (d, J=6 Hz, 3H), 2.98 (sep, J=6 Hz, 1H), 3.76-3.81 (m, 6H), 4.57 (d, J=12 Hz, 1H), 5.42 (d, J=12 Hz, 1H), 6.37-6.4 (m, 2H), 7.07 (t, J=9 Hz, 3H), 7.18-7.2 (m, 2H), 7.6-7.63 (m, 3H), 7.81 (d, J=9 Hz, 1H), 7.96-7.99 (m, 3H), 8.45 (brs, 1H), 8.58 (s, 1H); MS (Positive ion mode): m/z 479.25 (M⁺+1); Yield: 42.25%.

2-[2-(4-Fluorophenyl)-2-oxo-1-pyridin-4-yl-ethyl]-4-methyl-3-oxo-pentanoic acid (3-fluorophenyl) amide

¹H NMR (CDCl₃, 300 MHz): δ 1.15 (d, J=9 Hz, 3H), 1.24 (d, J=9 Hz, 3H), 2.97 (sep. J=9 Hz, 1H), 4.51 (d, J=9 Hz, 1H), 5.36 (d, J=9 Hz, 1H), 6.79-6.88 (m, 2H), 7.08 (t, J=9 Hz, 2H), 7.22 (d, J=6 Hz, 4H), 7.53 (s, 1H), 7.93-7.98 (m, 2H), 8.51 (d, J=6 Hz, 2H); MS (Positive ion mode): m/z 437.5 (M⁺+1); Yield: 22.12%.

2-[2-(4-Fluorophenyl)-2-oxo-1-pyridin-3yl-ethyl]-4methyl-3-oxo-pentanoic acid (4-methoxyphenyl) amide

¹H NMR (CDCl₃, 300 MHz): δ 0.96-0.99 (d, J=6 Hz, 3H), 1.08-1.10 (d, J=6 Hz, 3H), 2.99 (m, 1H), 3.75 (s, 3H), 4.59-4.62 (d, J=9 Hz, 1H), 5.42-5.46 (d, J=12 Hz, 1H), 6.74-6.77 (d, J=9 Hz, 2H), 7.04-7.10 (m, Ar—H, 4H), 7.22-7.26 (d, J=12 Hz, 2H), 7.5 (d, 1H), 7.96-7.99 (d, J=9 Hz, 2H), 8.47-8.49 (d, J=6 Hz, 1H), 8.52 (brs, 1H, —NH); MS (Positive ion mode): m/z 449 (M⁺+1); Yield: 44.85%.

2-[2-(4-Fluorophenyl)-2-oxo-1-pyridin-3-yl-ethyl]-4-methyl-3-oxo-pentanoic acid (3-fluorophenyl) amide

MS (Positive ion mode): m/z 437.6 (M⁺+1); Yield: 40.57%.

2-[2-(4-Fluorophenyl)-2-oxo-1-pyridin-3-yl-ethyl]-4methyl-3-oxo-pentanoic acid (2-benzyloxyphenyl) amide

MS (Positive ion mode): m/z 525.52 (M⁺+1); Yield: 47.6%.

2-[2-(4-Fluorophenyl)-2-oxo-1-pyridin-3-yl-ethyl]-4-methyl-3-oxo-pentanoic acid (2-methoxyphenyl) amide

¹H NMR (CDCl₃, 300 MHz): δ 1.05-1.07 (d, J=6 Hz, 3H), 1.12-1.14 (d, J=6 Hz, 3H), 2.98 (m, 1H), 3.81 (s, 3H), 4.58-4.62 (d, J=12 Hz, 1H), 5.41-5.45 (d, J=12 Hz, 1H), 6.8-8.57 (m, Ar—H, 12H); MS Positive ion mode): m/z 449 (M⁺+1).

2-[2-(4-Fluorophenyl)-2-oxo-1-pyridin-4-yl-ethyl]-4-methyl-3-oxo-pentanoic acid (4-methoxyphenyl) amide

MS Positive ion mode): m/z =449.45 [M⁺+1]; Yield: 65.8%.

2-[2-(4-Fluorophenyl)-2-oxo-1-pyridin-4-yl-ethyl]-4methyl-3-oxo-pentanoic acid (2-benzyloxyphenyl) amide

MS Positive ion mode) m/z 525.45 (M⁺+1); Yield: 52%.

2-[2-(4-Fluorophenyl)-2-oxo-1-pyridin-4-yl-ethyl]-4-methyl-3-oxo-pentanoic acid (2-methoxyphenyl) amide

¹H NMR (CDCl₃, 300 MHz): δ 1.13 (d, J=6 Hz, 3H), 1.18 (d, J=6 Hz, 3H),2.95 (sep, J=6.9 Hz, 1H), 3.80 (s, 3H), 4.56 (d, J=10.5 Hz, 1H), 5.41 (d, J=10.8 Hz, 1H), 6.81-6.92 (m, 3H), 7.05 (d, J=9 Hz, 3H), 7.31 (d, J=6 Hz, 2H), 7.96-8.01 (m, 3H), 8.48 (d, J=6 Hz, 2H); MS Positive ion mode): m/z 449.35 (M⁺+1); Yield: 87.7%.

2-[2-(3,4-Difluorophenyl)-2-oxo-1-thiophen-3-yl-ethyl]-4-methyl-3-oxo-pentanoic acid phenyl amide

¹H NMR (CDCl₃, 300 MHz): δ 1.08-1.10 (d, J=6 Hz, 3H), 1.13-1.15 (d, J=6 Hz, 3H), 2.9-2.95 (m, 1H), 4.47-4.50 (d, J=9 Hz, 1H), 5.45-5.48 (d, J=9 Hz, 1H), 6.98-7.78 (m, 10H); MS Positive ion mode): m/z 442 (M⁺+1); Yield: 37.29%.

Step 4: Preparation of Pyrrole (Formula X)

A mixture of diketone (Formula VIII, 1 equiv), amine (Formula IX, 1 equiv) and pivalic acid (1.03 equiv) in heptane: toluene: tetrahydrofuran (4:1:1) was refluxed and water trapped using Dean Stark trap. After the completion of reaction, solvents were removed and the residue was dissolved in ethyl acetate. The organic layer was washed in saturated sodium bicarbonate, water, dried over anhydrous sodium sulphate, concentrated by rotary evaporation and the residue was purified by column chromatography (silica gel, 100-200 mesh).

The following intermediates were prepared following above general procedure

(6-{2-[2-(4-Fluorophenyl)-5-isopropyl-3-phenyl-4-(5-methylthiazol-2-yl-amino)carbonyl]-pyrrol-1-yl]ethyl}-2,2-dimethyl-[1,3]dioxan-4-yl) acetic acid tert-butyl ester

MS (positive ion mode): m/z 676 (M⁺+1)

(6-{2-[2-(4-Fluorophenyl)-5-isopropyl-3-(pyridyl-2-yl)-4-(phenylamino)carbonyl]-pyrrol-1-yl]ethyl}-2,2-dimethyl-[1,3]dioxan-4-yl) acetic acid tert-butyl ester

¹H NMR (CDCl₃, 300 MHz): δ 0.90-1.05 (m, 1H), 1.28 (s, 3H), 1.35 (s, 3H), 1.43 (s, 9H), 1.54 (d, J=6 Hz, 6H), 2.22 (dd, J=15 & 6 Hz), 2.32 (dd, J=15 & 6 Hz, 1H), 3.61-3.65 (m, 2H), 3.85-4.00 (m, 1H), 4.15-4.25 (m, 2H), 6.77 (d, J=9 Hz, 1H), 6.97-7.16 (m, 7H), 7.25-7.34 (m, 4H), 7.62 (d, J=9 Hz, 2H), 8.62 (d, J=3 Hz, 1H), 10.72 (s, 1H); MS (positive ion mode): 656 (M⁺+1); Yield: 62%.

(6-{2-[2-(4-Fluorophenyl)-5-isopropyl-3-(pyridyl-3-yl)-4-phenylamino)carbonyl]-pyrrol-1-yl]ethyl}-2,2-dimethyl-[1,3]dioxan-4-yl) acetic acid tert-butyl ester

¹H NMR (CDCl₃): δ 1.30 (s, 3H), 1.36 (s, 3H), 1.43 (s, 9H), 1.51 (d, J=6 Hz, 6H), 1.13-1.81 (m, 3H), 2.24 (dd, J=15.3 & 6.3 Hz, 1H), 2.39 (dd, J=15.3 & 6.9 Hz, 1H), 3.42 (sept, J=6 Hz, 1H), 3.65-3.90 (m, 2H), 4.04-4.28 (m, 2H), 6.92-7.35 (m, 11H), 7.52 (d, 1H), 8.25-8.35 (m, 2H); MS (positive ion mode): m/z=656 (M⁺+1); Yield: 52%.

(6-{2-[2-(4-Fluorophenyl)-5-isopropyl-3-(pyridyl-4-yl)-4-(phenylamino)carbonyl]-pyrrol-1-yl]ethyl}-2,2-dimethyl-[1,3]dioxan-4-yl) acetic acid tert-butyl ester

¹H NMR (DMSO-d₆, 300 MHz): δ 0.87-0.89 (m, 1H), 1.16 (s, 3H), 1.31 (s, 3H), 1.38 (brs, 15H), 1.58 (brs, 2H), 3.77-4.04 (m, 5H), 6.93-7.04 (m, 3H), 7.22-7.30 (m, 6H), 7.54 (d, J=6 Hz, 2H), 8.23 (d, J=6 Hz, 2H), 10.03 (s, 1H); MS positive ion mode): m/z 656.5 (M⁺+1); Yield: 48%.

(6-{2-[2-(4-Fluorophenyl)-5-isopropyl-3-(5-methylfuran-2-yl)-4-(phenylamino)carbonyl]-pyrrol-1-yl]ethyl}-2,2-dimethyl-[1,3]dioxan-4-yl) acetic acid tert-butyl ester

¹H NMR (CDCl₃, 300 MHz): δ 0.89-1.15 (m, 2H), 1.28 (s, 3H), 1.35 (s, 3H), 1.43 (s, 9H), 1.49 (d, J=66 Hz, 6H), 1.56-1.63 (m, 2H), 2.10 (s, 3H), 2.21-2.37 (m, 2H), 3.35-3.65 (m, 1H), 3.65-3.85 (m, 2H), 3.95-4.05 (m, 2H), 5.79 (brs, 1H), 5.81 (brs, 1H), 7.02-7.10 (m, 2H), 7.20-7.30 (m, 4H), 7.41-7.44 (m, 2H), 7.58 (s, 1H); MS (positive ion mode): m/z 659.5 (M⁺+1); Yield: 54%.

(6-{2-[2-(4-Fluorophenyl)-5-isopropyl-3-(thiophen-2-yl)-4-(phenylamino)carbonyl]-pyrrol-1-yl]ethyl}-2,2-dimethyl-[1,3]dioxan-4-yl) acetic acid tert-butyl ester

MS (positive ion mode): m/z 661 (M⁺+1).

(6-{2-[2-(4-Fluorophenyl)-5-isopropyl-3-(thiophen-3-yl)-4-(phenylamino)carbonyl]-pyrrol-1-yl]ethyl}-2,2-dimethyl-[1,3]dioxan-4-yl) acetic acid tert-butyl ester

¹H NMR (CDCl₃): δ 1.30 (s, 3H), 1.36 (s, 3H), 1.43 (s, 9H), 1.51 (d, J=6.9 Hz, 6H), 2.20-2.32 (dd, J=15 & 9 Hz, H), 2.3-2.45 (dd, J=15.3 8.4 Hz, 1H), 3.55 (sept, J=6.9 Hz, 1H), 3.69 (brs, 1H), 3.77-3.87 (m, 1H), 4.00-4.22 (m, 2H), 6.85 (d, J=4.5 Hz, 1H), 6.94 (s, 1H), 7.03 (t, J=8.4 Hz, 3H), 7.13-7.30 (m, 8H); MS (positive ion mode): m/z 661 [M+1]; Yield: 23%.

(6-{2-[2-(4-Fluorophenyl)-5-isopropyl-3-(pyridin-3-yl)-4-(4-acetylphenylamino)carbonyl]-pyrrol-1-yl]ethyl}-2,2-dimethyl-[1,3]dioxan-4-yl) acetic acid tert-butyl ester

¹H NMR (300 MHz): δ 1.29 (s, 3H), 1.36 (s, 3H), 1.43 (s, 9H), 1.51 (d, J=6 Hz, 6H), 1.08-1.75 (m, 4H), 2.20-2.45 (m, 2H), 2.53 (s, 3H), 3.46 (sep, J=6.0 Hz, 1H), 3.63-3.91 (d, J=9 Hz, 1H), 4.04-4.23 (m, 2H), 6.95-7.35 (m, 8H), 7.49 (d, J=9 Hz, 1H), 7.83 (d, J=9 Hz, 2H), 8.29 (s, 1H), 8.35 (d, J=3 Hz, 1H); MS (Positive ion mode): m/z 699; Yield: 21.52%.

(6-{2-[2-(4-Fluorophenyl)-5-isopropyl-3-(thiophen-2-yl)-4-(3-fluorophenylamino)carbonyl]-pyrrol-1-yl]ethyl}-2,2-dimethyl-[1,3]dioxan-4-yl) acetic acid tert-butyl ester

¹H NMR (CDCl₃, 300 MHz): δ 0.98-1.06 (m, 1H), 1.26-1.29 (m, 4H), 1.36 (s, 3H), 1.43 (s, 9H), 1.51 (d, J=6 Hz, 6H), 1.61-1.68 (m, 2H), 2.25 (dd, J=6 & 9 Hz, 1H), 2.37 (dd, J=9 & 6 Hz, 1H), 3.55 (m, 1H), 3.59 (br s, 1H), 3.6-3.68 (m, 1H), 4.05 (m, 1H), 4.15 (brs, 1H), 6.7-6.74 (m, 2H), 6.86-6.93 (m, 2H), 7.01-7.29 (m, 8H); MS: (Positive ion mode): m/z 679.5 (M⁺+1); Yield: 71.58%.

(6-{2-[2-(4-Fluorophenyl)-5-isopropyl-3-(thiophen-3-yl)-4-(3-fluorophenylamino)carbonyl]-pyrrol-1-yl]ethyl}-2,2-dimethyl-[1,3]dioxan-4-yl) acetic acid tert-butyl ester

¹H NMR (CDCl₃, 300 MHz): δ 0.85-1.06 (m, 2H), 1.26 (s, 3H), 1.32 (s, 3H), 1.43 (s, 9H), 1.51 (d, J=6 Hz, 6H), 1.61-1.68 (m, 3H), 2.25 (dd, J=9 Hz, 1H), 2.36 (dd, J=9 Hz, 1H), 3.56-3.6 (m, 1H), 3.68 (brs, 1H), 3.75-3.9 (m, 1H), 4.06-4.17 (m, 2H), 6.68 (d, J=9 Hz, 2H), 6.85 (d, J=6 Hz, 1H), 6.94 (brs, 1H), 7.00-7.29 (m, 8H); MS (Positive ion mode): m/z 679.6 (M⁺+1); Yield: 68.04%.

(6-{2-[2-(4-Fluorophenyl)-5-isopropyl-3-(pyridin-4-yl)-4-(2,4-dimethoxyphenyl)amino)carbonyl]-pyrrol-1-yl]ethyl}-2,2-dimethyl-[1,3]dioxan-4-yl) acetic acid tert-butyl ester

¹H NMR (300 MHz): δ 1.03-1.16 (m, 2H), 1.30 (s, 3H), 1.36 (s, 3H), 1.43 (s, 9H), 1.49 (d, J=6.9 Hz, 6H), 1.61-1.81 (m, 2H), 2.24 (dd, J=6 & 15 Hz, 1H), 2.38 (dd, J=6.9 & 15 Hz, 1H), 3.40 (sep, J=6.9 Hz, 1H), 3.47 (s, 3H), 3.65-3.93 (m, 5H), 4.0-4.23 (m, 2H), 6.34 (s, 1H), 6.46 (d, J=7.8 Hz, 1H), 6.96-7.08 (m, 4H), 7.16-7.21 (m, 2H), 7.37 (s, 1H), 8.29 (d, J=6 Hz, 2H);

MS (Positive ion mode): m/z 716.70 (M⁺+1); Yield: 17.06%.

(6-{2-[2-(4-Fluorophenyl)-5-isopropyl-3-(pyridin-3-yl)-4-(2,4-dimethoxyphenylamino)carbonyl]-pyrrol-1-yl]ethyl}-2,2-dimethyl-[1,3]dioxan-4-yl) acetic acid tert-butyl ester

¹H NMR (CDCl₃, 300 MHz): δ 1.03-1.07 (m, 2H), 1.17-1.20 (m, 2H), 1.23 (s, 3H), 1.26 (s, 3H), 1.43 (s, 9H), 1.49 (d, J=6 Hz, 6H), 1.64-1.69 (m, 2H), 2.25 (dd, J=9 Hz, 1H), 2.36 (dd, J=9 Hz, 1H), 3.45-3.48 (m, 4H), 3.5-3.8 (m, 5H), 4.01-4.21 (m, 2H), 6.3 (s, 1H), 6.41-6.45 (m, 1H), 7.01-7.06 (m, 3H), 7.16-7.19 (m, 2H), 7.36 (s, 1H), 7.5 (d, J=7.8 Hz, 1H), 8.28-8.3 (m, 2H); MS (Positive ion mode): m/z 716.39 (M⁺+1); Yield: 52.58%.

(6-{2-[2-(4-Fluorophenyl)-5-isopropyl-3-(pyridin-4-yl)-4-(3-fluorophenylamino)carbonyl]-pyrrol-1-yl]ethyl}-2,2-dimethyl-[1,3]dioxan-4-yl) acetic acid tert-butyl ester

¹H NMR (CDCl₃, 300 MHz): δ 0.98-1.07 (m, 2H), 1.3 (s, 3H), 1.36 (s, 3H), 1.435 (s, 9H), 1.49 (d, J=6 Hz, 6H), 1.62-1.69 (m, 3H), 2.26 (dd, J=6.3 Hz, 1H), 2.36 (dd, J=6.3 Hz, 1H), 3.36-3.38 (m, 1H), 3.81 (m, 2H), 4.09-4.15 (m, 2H), 6.66-6.74 (m, 2H), 6.93-6.97 (m, 3H), 7.04-7.20 (m, 4H), 7.35 (brs, 1H), 8.32 (d, J=3 Hz, 2H); MS (Positive ion mode): m/z 674.8 (M⁺+1); Yield: 55.19%.

(6-{2-[2-(4-Fluorophenyl)-5-isopropyl-3-(pyridin-3-yl)-4-(4-methoxyphenyl)amino)carbonyl]-pyrrol-1-yl]ethyl}-2,2-dimethyl-[1,3]dioxan-4-yl) acetic acid tert-butyl ester

¹H NMR (CDCl₃, 300 MHz): δ 1.34-1.36 (d, 2H, J=6 Hz), 1.29 (d, 2H), 1.43 (s, 9H), 1.49 (s, 3H), 1.51 (s, 3H), 2.25-2.27 (dd, J=6 Hz, 1H), 2.35-2.37 (dd, J=6 Hz, 1H), 3.40 (m, 1H), 3.79 (s, 3H), 4.04-4.06 (d, J=6 Hz, 2H), 6.76-6.81 (m, 3H), 6.99-7.19 (m, ArH, 6H), 7.51-7.53 (d, J=6 Hz, 1H), 8.32-8.34 (d, J=6 Hz, 2H); MS (Positive ion mode): m/z: 686 (M⁺+1); Yield: 65.27%.

(6-{2-[2-(4-Fluorophenyl)-5-isopropyl-3-(pyridin-3-yl)-4-(3-fluorophenylamino)carbonyl]-pyrrol-1-yl]ethyl}-2,2-dimethyl-[1,3]dioxan-4-yl) acetic acid tert-butyl ester

¹H NMR (CDCl₃, 300 MHz): δ 1.03-1.17 (m, 3H), 1.27 (s, 3H), 1.36 (s, 6H), 1.43 (s, 9H), 1.5 (d, J=6 Hz, 6H), 1.62-1.67 (m, 2H), 2.27 (dd, J=6 Hz, 1H), 2.37 (dd, J=6 Hz, 1H), 3.4 (m, 1H), 3.68-3.77 (m, 2H), 4.02-4.14 (m, 2H), 6.67-6.74 (m, 2H), 6.98-7.27 (m, 9H), 7.5 (d, J=6 Hz, 1H), 8.22 (s, 1H), 8.31 (d, J=6 Hz, 1H); MS (Positive ion mode): m/z 674.43 (M⁺+1); Yield: 70.27%.

(6-{2-[2-(4-Fluorophenyl)-5-isopropyl-3-(pyridin-3-yl)-4-(2-benzyloxyphenyl)amino)carbonyl]-pyrrol-1-yl]ethyl}-2,2-dimethyl-[1,3]dioxan-4-yl) acetic acid tert-butyl ester

¹H NMR (CDCl₃, 300 MHz): δ 1.0-1.16 (m, 2H), 1.30 (s, 3H), 1.37 (s, 3H), 1.44 (s, 9H), 1.48 (d, J=9.0 Hz, 6H), 1.55-1.70 (m, 2H), 6.60-6.80 (m, 1H), 6.92-7.05 (m, 5H), 7.14-7.15 (m, 4H), 7.31-7.36 (m, 3H), 7.40-7.50 (m, 1H), 7.66 (s, 1H), 8.26 (d, J=3 Hz, 2H), 8.55-8.65 (m, 1H); MS (Positive ion mode): m/z 762.71 (M⁺+1); Yield: 30.55%.

(6-{2-[2-(4-Fluorophenyl)-5-isopropyl-3-(pyridin-3-yl)-4-(2-methoxyphenyl)amino)carbonyl]-pyrrol-1-yl]ethyl}-2,2-dimethyl-[1,3]dioxan-4-yl) acetic acid tert-butyl ester

¹H NMR (CDCl₃, 300 MHz): δ 1.30 (s, 3H), 1.39 (s, 3H), 1.43 (s, 9H), 1.50-1.52 (d, J=6 Hz, 6H), 2.26 (dd, J=6 Hz, 1H), 2.36 (dd, J=6 Hz, 1H), 3.44 (m, 1H), 3.51 (s, 3H), 4.08 (m, J=6 Hz, 2H), 6.69-6.72 (d, J=9 Hz, 2H), 6.93-7.26 (m, Ar—H, 6H), 7.59 (s, 2H), 8.29-8.31 (d, J=6 Hz, 2H), 8.32 (brs, 1H, —NH); MS (Positive ion mode): m/z 686 (M⁺+1); Yield: 78.2%.

(6-{2-[2-(4-Fluorophenyl)-5-isopropyl-3-(pyridin-4-yl)-4(4-methoxyphenyl)amino)carbonyl]-pyrrol-1-yl]ethyl}-2,2-dimethyl-[1,3]dioxan-4-yl) acetic acid tert-butyl ester

¹H NMR (300 MHz): δ 1.02-1.15 (m, 2H), 1.30 (s, 3H), 1.37 (s, 3H), 1.43 (s, 9H), 1.50 (d, J=6 Hz, 6H), 1.60-1.75 (m, 2H), 2.23-2.32 (m, 1H), 2.35-2.44 (m, 1H), 3.34-3.36 (m, 1H), 3.7-3.85 (m, 5H), 4.0-4.25 (m, 2H), 6.78-6.98 (m, 3H), 7.03-7.20 (m, 7H), 8.33 (d, J=6 Hz, 2H).

MS (Positive ion mode): m/z=686.66 [M⁺+1]; Yield: 58%.

(6-{2-[2-(4-Fluorophenyl)-5-isopropyl-3-(pyridin-4-yl)-4-(2-benzyloxyphenylamino)carbonyl]-pyrrol-1-yl]ethyl}-2,2-dimethyl-[1,3]dioxan-4-yl) acetic acid tert-butyl ester

¹H NMR (300 MHz): δ 1.00-1.20 (m, 2H), 1.29 (s, 3H), 1.36 (s, 3H), 1.43 (s, 9H), 1.47 (d, J=9.0 Hz, 6H), 1.60-1.87 (m, 2H), 2.20-2.27 (m, 1H), 2.37-2.43 (m, 1H), 3.35 (sep, J=6 Hz, 1H), 3.6-3.9 (m, 2H), 3.97-4.25 (m, 2H) 4.81 (s, 2H), 6.75-7.20 (m, 12H), 7.25-7.45 (m, 3H), 7.66 (s, 1H), 8.20 (d, J=6 Hz, 2H); MS (Positive ion mode) m/z 762.67 (M⁺+1).

(6-{2-[2-(4-Fluorophenyl)-5-isopropyl-3-(pyridin-4-yl)-4-(2-methoxyphenyl)amino)carbonyl]-pyrrol-1-yl]ethyl}-2,2-dimethyl-[1,3]dioxan-4-yl) acetic acid tert-butyl ester

¹H NMR (300 MHz): δ 1.0-1.20 (m, 2H), 1.30 (s, 3H), 1.39 (s, 3H), 1.43 (s, 9H), 1.52 (d, J=6 Hz, 6H), 1.60-1.70 (m, 2H), 2.24 (dd, J=6.9 & 12 Hz, 1H), 2.39 (dd, J=6.9 & 12.0 Hz, 1H), 3.40 (sep, J=7.2 Hz, 1H), 3.51 (s, 3H), 3.65-3.85 (m, 2H), 4.0-4.19 (m, 2H), 6.74 (d, J=6 Hz, 1H), 6.96-7.10 (m, 6H), 7.17-7.22 (m, 2H), 7.61 (brs, 1H), 8.28 (d, J=6 Hz, 2H), 8.45 (brd, J=9 Hz, 1H); MS (Positive ion mode): m/z 686.61 (M⁺+1); Yield: 66.2%.

(6-{2-[2-(3,4-Difluorophenyl)-5-isopropyl-3-(thiophen-3-yl)-4-phenylamino)carbonyl]-pyrrol-1-yl]ethyl}-2,2-dimethyl-[1,3]dioxan-4-yl) acetic acid tert-butyl ester

¹H NMR (CDCl₃, 300 MHz): δ 1.29 (s, 3H), 1.37 (s, 3H), 1.43 (s, 9H), 1.51 (d, J=6 Hz, 6H), 1.60-1.78 (m, 2H), 2.23-2.48 (m, 2H), 3.54 (sep, J=6 Hz, 1H), 3.65-3.90 (m, 2H), 4.00-4.28 (m, 2H), 6.83-7.30 (m, 12H); Yield: 67%.

Step 4-A: Preparation of Pyrrole (Formula X-A, when R₄ or R₅ is 2-hydroxyphenyl)

To a solution of a compound of Formula X (when R₄ or R₅ is 2-benzyloxyphenyl) (0.8 g) in methanol:dioxan (2:8) mixture was added 10% palladium carbon (50% wet, 60% w/w). The resulting reaction mixture was hydrogenated at 40 psi for about 2.5 hours. After the reaction was over, the reaction mixture was passed through celite and the resulting solution was concentrated under vacuum to give the required product.

(6- {2-[2- (4-Fluorophenyl)-5-isopropyl-3-(pyridyl-3-yl)-4-(2-hydroxyphenylamino) carbonyl]-pyrrol-1-yl]ethyl}-2,2-dimethyl-[1,3]dioxan-4-yl) acetic acid tert-butyl ester

¹H NMR (DMSO-d_{6,} 300 MHz): δ 1.05-1.15 (m, 2H), 1.30 (s, 3H), 1.32 (s, 3H), 1.43 (s, 9H),1.52 (d, J=6 Hz, 6H), 1.65-1.80 (m, 2H), 6.16 (d, J=6 Hz, 1H), 6.67 (t, J=6 Hz, 1H), 6.96-7.06 (m, 4H), 7.15-7.20 (m, 3H), 7.53 (d, J=6 Hz, 1H), 8.30-8.40 (m, 2H); MS (positive ion mode): m/z 672.62 (M⁺+1); Yield: 76%.

(6-{2-[2-(4-Fluorophenyl)-5-isopropyl-3-(pyridyl-4-yl)-4-(2-hydroxyphenylamino)carbonyl]-pyrrol-1-yl]ethyl}-2,2-dimethyl-[1,3]dioxan-4-yl) acetic acid tert-butyl ester

¹H NMR (CDCl₃, 300 MHz): δ 1.05-1.20 (m, 2H), 1.30 (s, 3H), 1.32 (s, 3H), 1.43 (s, 9H), 1.52 (d, J=6 Hz, 6H), 1.65-1.75 (m, 2H), 2.20-2.27 (m, 1H), 2.36-2.43 (m, 1H), 3.42 (sep, J=6 Hz, 1H), 3.65-3.95 (m, 2H), 4.02-4.30 (m, 2H), 6.46 (d, J=6 Hz, 1H), 6.71 (t, J=6 Hz, 1H), 7.0-7.09 (m, 7H), 7.17-7.21 (m, 2H), 8.33 (d, J=3 Hz, 2H); MS (positive ion mode): m/z 672.63 (M⁺+1); Yield: 57%.

Step 5: Preparation of Hemi Calcium Salt of Formula XI

(a) To a solution of a compound of Formula X or X-A in methanol and tetrahydrofuran (1:1) was added 1N hydrochloric acid (3 equiv) and the mixture stirred at ambient temperature. After the complete hydrolysis of the ketal, the reaction mixture was cooled to 0° C. and sodium hydroxide pellets (6 equiv) were added. The reaction was then stirred at ambient temperature. At the end of ester hydrolysis, solvents were removed and, the residue was dissolved in water; aqueous layer was washed with ether, and neutralized with 1N hydrochloric acid. The organic phase was extracted into ethyl acetate, and concentrated. The residue was then purified on a chromatographic column (silica gel 100-200 mesh).

(b) To an aqueous solution of sodium salt of acid (prepared by adding 1 equivalent 1N sodium hydroxide solution) was added dropwise an aqueous solution (1M) of calcium acetate (0.55 equiv). White precipitate was obtained, which was filtered off, washed with copious amout of water, and dried in vacuo.

The following compounds, were prepared following above general procedure.

Hemi Calcium Salt of (3R,5R)-7-[2-(4-Fluorophenyl)-5-isopropyl-3-phenyl-4-(4-methylthiazol-2-ylamino)carbonyl]-pyrrol-1-yl]-3,5-dihydroxy-heptanoic acid

Hemi Calcium Salt of (3R,5R)-7-[2-(4-Fluorophenyl)-5-isopropyl-3-(pyridin-2-yl)-4-(phenylamino)carbonyl]-pyrrol-1-yl]-3,5-dihydroxy-heptanoic acid

¹H NMR (DMSO-d₆, 300 MHz): δ 1.20-1.24 (m, 2H), 1.40 (d, J=6 Hz, 6H), 1.52-1.59 (m, 2H), 1.92-1.98 (m, 1H), 2.06-2.11 (m, 1H), 3.52 (brs, 2H), 3.75 (brs, 2H), 3.97 (brs, 1H), 6.85 (d, J=9 Hz, 1H), 6.97-7.04 (m, 2H), 7.17-7.30 (m, 6H), 7.44 (t, 6 Hz, 1H), 7.55 (d, J=6 Hz, 2H), 8.41 (brs, 1H), 10.28 (s, 1H); MS (positive ion mode): m/z 560 (Acid+1); Yield: 23%; m.p.: 165-200° C.

Hemi Calcium Salt of (3R,5R)-7-[2-(4-Fluorophenyl)-5-isopropyl-3-(pyridin-3-yl)-4-(phenylamino)carbonyl]-pyrrol-1-yl]-3,5-dihydroxy-heptanoic acid

¹H NMR (DMSO-d₆,): δ 1.24 (brs, 2H), 1.38 (d, J=9 Hz, 6H), 1.53 (brs, 2H), 1.87-2.13 (m, 2H), 3.23 (brs, 1H), 3.50-3.75 (brs, 1H), 3.97 (brs, 1H), 6.99 (t, J=6 Hz, 1H), 7.05-7.37 (m, 7H), 7.41 (d, J=9 Hz, 1H), 7.52 (d, J=6 Hz, 2H), 8.19 (d, J=6 Hz, 2H), 9.98 (s, 1H, D₂O exchanged); MS (positive ion mode): m/z 560 [Acid+1]; Yield: 50%; m.pt.: 196-221° C.

Hemi Calcium Salt of (3R,5R)-7-[2-(4-Fluorophenyl)-5-isopropyl-3-(pyridin-4-yl)-4-(phenylamino)carbonyl]-pyrrol-1-yl]-3,5-dihydroxy-heptanoic acid

¹H NMR (DMSO-d₆, 300 MHz): δ 1.18-1.24 (m, 1H), 1.37 (d, J=6 Hz, 6H), 1.53-1.58 (m, 2H), 1.90 (dd, J=15 & 6H, 1H), 2.02-2.06 (m, 1H), 3.51 (brs, 2H), 3.72 (brs, 2H), 4.00 (brs, 1H), 6.93-7.04 (m, 3H), 7.22-7.30 (m, 6H), 7.56 (d, J=9 Hz, 2H), 8.22 (d, J=5 Hz, 2H), 10.08 (s, 1H)

MS (positive ion mode): m/z 560.8 (Acid +1); Yield: 35%; m.p.: 170° C.-244° C.

Hemi Calcium Salt of (3R,5R)-7-[2-(4-Fluorophenyl)-5-isopropyl-3-(5-methylfuran-2-yl)-4-phenylamino)carbonyl]-pyrrol-1-yl]-3,5-dihydroxy-heptanoic acid

¹H NMR (DMSO-d₆, 300 MHz): δ 1.28 (d, J=6 Hz, 6H), 1.27-1.52 (m, 4H), 1.86 (s, 3H), 1.95-2.02 (m, 2H), 3.13 (brs, 1H), 3.45 (brs, 1H), 3.67 (brs, 2H), 3.85 (brs, 1H), 5.59 (s, 1H), 5.77 (s, 1H), 7.02-7.05 (m, 1H), 7.19-7.29 (m, 6H), 7.49 (d, J=7.6 Hz, 2H); MS (positive ion mode): m/z 563 (Acid+1); Yield: 14%; m.p.: 145-211° C. (Dec.).

Hemi Calcium Salt of (3R,5R)-7-[2-(4-Fluorophenyl)-5-isopropyl-3-(thiophen-2-yl)-4-(phenylamino)carbonyl]-pyrrol-1-yl]-3,5-dihydroxy-heptanoic acid

¹H NMR (DMSO-d₆, 300 MHz): δ 1.28 (d, J=6.2 Hz, 6H), 1.27-1.34 (m, 4H), 1.95-2.05 (m, 2H), 3.14 (m, 1H), 3.45 (brs, 1H), 3.67 (brs, 2H), 3.84 (m, 1H), 6.69 (brs, 1H), 6.75 (brs, 1H), 7.02-7.09 (m, 2H), 7.17-7.27 (m, 6H), 7.48-7.51 (m, 2H); MS (positive ion mode): m/z 564 (acid+1).

Hemi Calcium Salt of (3R,5R)-7-[2-(4-Fluorophenyl)-5-isopropyl-3-(thiophen-3-yl)-4-(phenylamino)carbonyl]-pyrrol-1-yl]-3,5-dihydroxy-heptanoic acid

¹H NMR (DMSO): δ 1.23-1.60 (m, 8H), 1.66 (brs, 2H), 2.17-2.38 (m, 2H), 3.65 (brs, 1H), 3.85-3.99 (m, 1H), 4.02 (brs, 2H), 6.77 (d, J=4.3 Hz, 1H), 6.94 (s, 1H), 7.10 (t, J=8.8 Hz, 4H), 7.19-7.42 (m, 6H); MS positive ion mode): m/z 566 [Acid+1]; Yield: 4%; m.p.: 197-213° C.

Hemi Calcium Salt of (3R,5R)-7-[2-(4-Fluorophenyl)-5-isopropyl-3-(pyridin-3-yl)-4-(4-acetylphenylamino)carbonyl]-pyrrol-1-yl]-3,5-dihydroxy-heptanoic acid

¹H NMR (DMSO-d₆, 300 MHz): δ 1.20-1.75 (m, 10H), 1.92 (dd, J=9 & 15 Hz, 1H), 2.06 (dd, J=9.0 & 15 Hz, 1H), 3.75-3.90 (m, 3H), 3.95-4.15 (m, 2H), 7.08-7.41 (m, 6H), 7.65 (d, J=6.0 Hz, 2H), 7.84 (d, J=9 Hz, 2H), 8.19 (s, 2H), 10.33 (s, 1H); MS (Positive ion mode): m/z 602.8 [Acid+1]; m.p.: 199.4-223.6° C.

Hemi Calcium Salt of (3R,5R)-7-[2-(4-Fluorophenyl)-5-isopropyl-3-(thiophen-2-yl)-4-(3-fluorophenyl amino)carbonyl]-pyrrol-1-yl]-3,5-dihydroxy-heptanoic acid

¹H NMR (DMSO-d₆, 300 MHz): δ 1.23 (brs, 3H), 1.33 (d, J=9 Hz, 6H), 1.54 (brs, 2H), 1.87-2.07 (m, 2H), 3.18-3.22 (m, 1H), 3.37 (brs, 1H), 3.73 (brs, 2H), 3.91 (brs, 1H1), 6.72-6.87 (m, 3H), 7.15 (d, J=6 Hz, 1H), 7.22-7.38 (m, 6H), 7.61 (d, J=12 Hz, 1H), 10.36 (s, 1H); MS (Positive ion mode): m/z 583.7 (Acid+1); Yield: 78%.

Hemi Calcium Salt of (3R,5R)-7-[2-(4-Fluorophenyl)-5-isopropyl-3-(thiophen-3-yl)-4-(3-fluorophenyl amino)carbonyl]-pyrrol-1-yl]-3,5-dihydroxy-heptanoic acid

¹H NMR (DMSO-d₆, 300 MHz): δ 1.19-1.24 (m, 2H), 1.34 (d, J=6 Hz, 6H), 1.5 (brs, 2H), 1.95 (dd, J=6 & 15 Hz, 1H), 2.07 (dd, J=6 & 15 Hz, 1H), 3.2 (m, 1H), 3.51 (brs, 1H), 3.74 (brs, 2H), 3.93 (m, 1H), 6.67 (d, J=6 Hz, 1H), 6.83 (t, J=9 Hz, 1H), 6.92 (s, 1H), 7.2-7.35 (m, 7H), 7.59 (d, J=12 Hz, 1H), 10.21 (s, 1H); MS (Positive ion mode): m/z 583.5 (Acid+1); Yield: 71.38%.

Hemi Calcium Salt of (3R,5R)-7-[2-(4-Fluorophenyl)-5-isopropyl-3-(pyridin-4-yl)-4-(2,4-dimethoxyphenyl amino)carbonyl]-pyrrol-1-yl]-3,5-dihydroxy-heptanoic acid

¹H NMR (DMSO-d₆, 300 MHz): δ 1.18-1.20 (m, 2H), 1.25-1.75 (m, 8H), 1.79-2.00 (m, 1H), 2.03-2.17 (m, 1H), 3.57 (s, 3H), 3.68-3.90 (m, 5H), 3.95-4.15 (m, 2H), 6.45-6.51 (m, 2H), 6.95-7.10 (m, 2H), 7.20-7.43 (m, 3H), 7.67 (d, J=9 Hz, 2H), 8.26-8.35 (m, 2H); MS (Positive ion mode): m/z 620.53 (Acid+1); Yield: 22.67%.

Hemi Calcium Salt of (3R,5R)-7-[2-(4-Fluorophenyl)-5-isopropyl-3-(pyridin-3-yl)-4-(2,4-dimethoxyphenyl amino)carbonyl]-pyrrol-1-yl]-3,5-dihydroxy-heptanoic acid

¹H NMR (DMSO-d₆, 300 MHz): δ 1.23 (brs, 2H), 1.41 (d, J=6 Hz, 6H), 1.58-1.6 (m, 2H), 1.93-2.04 (m, 2H), 3.54 (brs, 5H), 3.7 (brs, 5H), 3.96 (brs, 1H), 6.42-6.48 (m, 2H), 7.17-7.2 (m, 2H), 7.28 (brs, 2H), 7.44 (d, J=6 Hz, 1H), 7.7 (d, J=6 Hz, 1H), 8.12 (s, 1H), 8.22-8.28 (m, 2H); MS (Positive ion mode): m/z 620.33 (Acid+1); Yield: 47.88%.

Hemi Calcium Salt of (3R,5R)-7-[2-(4-Fluorophenyl)-5-isopropyl-3-(pyridin-4-yl)-4-(3-fluorophenyl amino)carbonyl]-pyrrol-1-yl]-3,5-dihydroxy-heptanoic acid

¹H NMR (DMSO-d₆, 300 MHz): δ 1.24 (brs, 2H), 1.36 (d, J=6 Hz, 6H), 1.6 (brs, 2H), 1.94-2.11 (m, 2H), 3.53 (brs, 2H), 3.78 (brs, 2H), 3.94-3.96 (m, 1H), 6.83 (brs, 1H), 6.92 (d, J=6 Hz, 2H), 7.21-7.29 (m, 6H), 7.54 (d, J=12 Hz, 1H), 8.22 (d, J=6 Hz, 2H), 10.26 (s, 1H); MS (Positive ion mode): m/z 578.26 (Acid+1); Yield: 46.3%.

Hemi Calcium Salt of (3R,5R)-7-[2-(4-fluorophenyl)-5-isopropyl-3-(pyridin-3-yl)-4-(4-methoxyphenyl amino)carbonyl]-pyrrol-1-yl]-3,5-dihydroxy-heptanoic acid

¹H NMR (CDCl₃, 300 MHz): δ 1.23 (brs, 2H), 1.36-1.38 (d, J=6 Hz, 6H), 1.53-1.57 (d, J=12 Hz, 2H), 1.91-2.01 (dd, J=6 Hz, 2H), 3.32 (s, 2H), 3.51 (m, 1H), 3.69 (s, 3H), 6.79-6.82 (d, J=9 Hz, 2H), 7.10-7.27 (Ar—H, 6H), 7.40-7.43 (d, J=9 Hz, 2H), 8.2 (s, 2H), 9.8 (brs, 1H, N H); Yield: 29.14%.

Hemi Calcium Salt of (3R,5R)-7-[2-(4-fluorophenyl)-5-isopropyl-3-(pyridin-3-yl)-4-(3-fluorophenyl amino)carbonyl]-pyrrol-1-yl]-3,5-dihydroxy-heptanoic acid

¹H NMR (DMSO-d₆, 300 MHz): δ 1.22-1.26 (m, 2H), 1.37 (d, J=6 Hz, 6H), 1.5 (brs, 2H), 1.91-2.11 (m, 2H), 3.53 (brs, 2H), 3.77 (brs, 2H), 3.97 (m, 1H), 6.81 (brs, 1H), 7.11-7.14 (m, 1H), 7.18-7.31 (m, 6H), 7.39 (d, J=6 Hz, 1H), 7.51 (d, J=12 Hz, 1H), 8.19 (s, 2H), 10.18 (s, 1H); MS (Positive ion mode): m/z 578.36 (Acid+1); Yield: 56.15%.

Hemi Calcium Salt of (3R,5R)-7-[2-(4-fluorophenyl)-5-isopropyl-3-(pyridin-3-yl)-4-(2-hydroxyphenyl amino)carbonyl]-pyrrol-1-yl]-3,5-dihydroxy-heptanoic acid

¹H NMR (DMSO-d₆, 300 MHz): δ 1.10-1.25 (m, 2H), 1.39 (d, J=3 Hz, 6H), 1.5-1.7 (m, 2H), 3.6-3.85 (m, 3H), 3.95-4.15 (m, 1H), 6.60-6.70 (m, 2H), 6.75-6.85 (m, 1H), 7.05-7.20 (m, 3H), 7.25-7.35 (m, 2H), 7.40-7.55 (m, 1H), 7.60-7.70 (m, 1H), 8.22 (brs, 1H); MS (.Positive ion mode): m/z 576.45 (Acid+1); Yield: 5.8%.

Hemi Calcium Salt of (3R,5R)-7-[2-(4-fluorophenyl)-5-isopropyl-3-(pyridin-3-yl)-4-(2-methoxyphenyl amino)carbonyl]-pyrrol-1-yl]-3,5-dihydroxy-heptanoic acid

¹H NMR (DMSO-d₆, 300 MHz): δ 1.15-1.25 (m, 2H), 1.43 (d, J=6 Hz, 6H), 1.55-1.70 (m, 2H), 3.38 (s, 3H), 3.70-3.83 (m, 3H), 3.90-4.10 (m, 1H), 6.88 (d, J=9 Hz, 2H), 6.98 (d, J=9 Hz, 1H), 7.16-7.23 (m, 2H), 7.29-7.34 (m, 2H), 7.45-7.5 (m, 1H), 7.90-8.00 (m, 1H), 8.12 (s, 1H), 8.24 (d, J=9 Hz, 2H); MS (Positive ion mode): m/z 590.55 (Acid+1); Yield: 52.39%.

Hemi Calcium Salt of (3R,5R)-7-[2-(4-fluorophenyl)-5-isopropyl-3-(pyridin-4-yl)-4-(4-methoxyphenyl amino)carbonyl]-pyrrol-1-yl]-3,5-dihydroxy-heptanoic acid

¹H NMR (DMSO-d₆, 300 MHz): δ 1.20-1.30 (m, 2H), 1.36 (d, J=6.6 Hz, 6H), 1.5-1.7 (m, 2H), 1.85-2.20 (m, 2H), 3.70 (s, 3H), 6.83 (d, J=4.3 Hz, 2H), 6.94 (d, J=4.8 Hz, 2H), 7.21-7.29 (m, 4H), 7.44 (d, J=8.7 Hz, 2H), 8.22 (d, J=4.8 Hz, 2H), 9.9 (s, 1H); MS (Positive ion mode): m/z 590.48 (Acid+1); Yield: 11.29%.

Hemi Calcium Salt of (3R,5R)-7-[2-(4-fluorophenyl)-5-isopropyl-3-(pyridin-4-yl)-4-(2-hydroxyphenyl amino)carbonyl]-pyrrol-1-yl]-3,5-dihydroxy-heptanoic acid

¹H NMR (DMSO-d₆, 300 MHz): δ 1.10-1.20 (m, 2H), 1.24-1.49 (m, 6H), 1.5-1.75 (m, 2H), 1.95-2.20 (m, 2H), 3.5-3.7 (m, 2H), 3.75-3.90 (m, 2H), 3.95-4.15 (m, 1H), 6.69-6.78 (m, 2H), 6.88-6.93 (m, 1H), 6.99 (d, 5.1 Hz, 2H), 7.22 (t, J=8.7 Hz, 2H), 7.3-7.34 (m, 2H), 7.63 (d, J=7.8 Hz, 1H), 8.25 (d, J=5.4 Hz, 2H), 9.13 (s, 1H); Yield: 26.3%.

Hemi Calcium Salt of (3R,5R)-7-[2-(4-fluorophenyl)-5-isopropyl-3-(pyridin-4-yl)-4-(2-methoxyphenyl amino)carbonyl]-pyrrol-1-yl]-3,5-dihydroxy-heptanoic acid

¹H NMR (CDCl₃, 300 MHz): δ 1.10-1.25 (m, 2H), 1.41 (d, J=9 Hz, 6H), 1.6-1.75 (m, 2H), 1.80-2.10 (m, 2H), 3.60-3.80 (m, 3H), 3.85-4.10 (m, 2H), 6.86-7.03 (m, 5H), 7.19-7.34 (m, 4H), 7.90-8.10 (m, 1H), 8.26-8.30 (m, 3H); MS (Positive ion mode): 590.48 (Acid+1); Yield: 16.6%.

Hemi Calcium Salt of (3R,5R)-7-[2-(3,4-difluorophenyl)-5-isopropyl-3-(thiophen-3-yl)-4-(phenyl amino)carbonyl]-pyrrol-1-yl]-3,5-dihydroxy-heptanoic acid

¹H NMR (DMSO-d₆, 300 MHz): δ 1.22-1.70 (m, 10H), 1.90-2.15 (m, 2H), 3.18-3.63 (m, 2H), 3.72-3.90 (brm, 2H), 3.91-4.15 (brm, 1H), 6.73 (d, J=3 Hz, 1H), 7.00-7.20 (m, 3H), 7.25-7.40 (m, 4H), 7.41-7.55 (m, 1H), 7.59 (d, J=9 Hz, 2H), 9.98 (s, 1H, D₂O exchanged); MS (Positive ion mode): m/z 584 (Acid+1); m.pt: 178.2-204° C.; Yield: 31.51%.

Scheme II

The compounds disclosed herein can also be prepared following the procedures described in Scheme II.

Preparation of Compound of Formula XIV

To a solution of a compound of Formula XIII (1 equiv.; prepared according to analogous procedures as for Scheme I) in toluene (15 ml) was added a compound of Formula V (1.08 equiv.), piperidine and acetic acid. The mixture was heated at reflux with azeotropic removal of water for about 4 to 6 hours. The reaction mixture was concentrated and the residue was extracted in dichloromethane. The organic layer was washed with 1N hydrochloric acid solution, sodium bicarbonate solution, brine, dried over anhydrous sodium sulphate, and concentrated. The crude product was purified on a chromatographic column (silica gel, 100-200 mesh).

2-Benzylidene-4-methyl-3-oxo-pentanoic acid benzyl ester

Preparation of Compound of Formula XV

A compound of Formula XIV (1 equiv.), a compound of Formula VII (1.104 equiv.), 3-ethyl-5-(2-hydroxyethyl)-4-methyl thiazolium bromide (0.2 equiv.), triethyl amine (1 equiv.) and ethanol were placed in a 30 ml vial, flushed with argon and the vial was sealed properly. The reaction mixture was stirred at 70° C. for about 12 to 15 hours. To the reaction mixture was added ethyl acetate, the mixture was washed with water, 6N hydrochloric acid, again with water and brine, dried over anhydrous sodium sulphate, and concentrated to give crude product. The crude product was purified on a chromatographic column (silica gel 100-200 mesh).

2-[2-(4-Fluorophenyl)-2-oxo-1-phenyl-ethyl]-4-methyl-3-oxo-pentanoic acid benzyl ester

Preparation of Compound of Formula XVI

To a solution of Formula XV (1 equiv.) in heptane:toluene:tetrahydrofuran (4:1:1) were added a compound of Formula IX (1.51 equiv.) and pivalic acid (1.03 equiv.). The mixture was refluxed with azeotropic removal of water for about 22 to 25 hours. The reaction mixture was concentrated, added ethyl acetate, washed with sodium bicarbonate solution and brine, dried over anhydrous sodium sulphate and concentrated to give the crude product. The crude product was purified on column (silica gel, 100-200 mesh).

1-[2-(6-Tert-butoxycarbonylmethyl-2,2-dimethyl-[1,3]dioxan-4-yl)-ethyl]-2-(4-fluorophenyl)-5-isopropyl-3-phenyl-1H-pyrrole-3-carboxylic acid benzyl ester

Preparation of Compound of Formula XVII

To a solution of a compound of Formula XVI (1 equiv.) in methanol:dioxan (2:8) mixture was added 10% palladium carbon (50% wet, 60% w/w). The resulting reaction mixture was hydrogenated at 40 psi for about 2.5 hours. After the reaction was over, the reaction mixture was passed through celite and the resulting solution was concentrated under vacuum to give the required product, which was further used as such for next step.

1-[2-(6-Tert-butoxycarbonylmethyl-2,2-dimethyl-[1,3]dioxan-4-yl)-ethyl]-2-(4-fluorophenyl)-5-isopropyl-3-phenyl-1H-pyrrole-3-carboxylic acid

Preparation of Compound of Formula X: Path a

To a solution of a compound of Formula XVII (1 equiv) in benzene at 0° C. under argon, oxalyl chloride (2.0 equiv) is added dropwise. After the evolution of gas had ceased, the reaction mixture is heated on oil bath at 70° C. for about 2 hours. The reaction mixture is evaporated to dryness. The residue is dissolved in benzene (dry) and added at ambient temperature to a solution of amine of formula III (1.1 equiv.) in benzene. The reaction mixture is then heated to 70° C. until completion of reaction. Volatiles are removed in vacuo and the residue is purified on a chromatographic column (silica gel, 100-200 mesh).

Preparation of Compound of Formula X: Path b

To a solution of a compound of Formula XVII (1 equiv.) in dimethylformamide was added diisopropylethylamine (2 equiv.) and O-benzotriazol-1-yl-N,N,N′,N′-tetramethyl uronium hexafluorophosphate (HBTU) (1 equiv.). To the resulting clear solution was then added cyclohexylamine (1 equiv.) in dimethylformamide, the reaction mixture was stirred at 50° C. to 60° C. overnight. To the reaction mixture was added water and the mixture was extracted with dicloromethane, the organic layer was washed with water, brine, dried over anhydrous sodium sulphate and concentrated to get the crude product. The crude product was purified by column chromatography (silica gel, 100-200 mesh).

The following compound was prepared as per this protocol.

(6-{2-[2-(4-Fluorophenyl)-5-isopropyl-3-phenyl-4-(1H-indol-5-yl-amino)carbonyl]-pyrrol-1-yl]ethyl}-2,2-dimethyl-[1,3]dioxan-4-yl) acetic acid tert-butyl ester

¹H NMR (CDCl₃, 300 MHz): δ 1.28 (s, 3H), 1.36 (s, 3H), 1.43 (s, 9H), 1.55 (d, J=7.2 Hz, 6H), 1.65-1.70 (m, 2H), 2.24 (dd, J=15 & 6 Hz, 1H), 2.37 (dd, J=15 & 6 Hz, 1H), 3.49-3.54 (m, 1H), 3.60-3.95 (m, 2H), 4.10-4.30 (m, 2H), 6.44 (brs, 1H), 6.71 (d, J=8.7 Hz, 1H), 6.90 (s, 1H), 6.99 (t, J=8.4 Hz, 2H), 7.13-7.20 (m, 9H), 7.58 (s, 1H), 8.11 (s, 1H); MS (positive ion mode): m/z 694 (M⁺+1); Yield: 54%.

Preparation of Hemi Calcium Salt of Formula XI

To a solution of a compound of Formula X in methanol and tetrahydrofuran (1:1) was added 1N hydrochloric acid (3 equiv) and the mixture stirred at ambient temperature. After the complete hydrolysis of ketal, the reaction mixture was cooled to 0° C. and sodium hydroxide pellets (6 equiv) were added. The reaction was then stirred at ambient temperature. At the end of ester hydrolysis, solvents were removed and the residue was dissolved in water; the aqueous layer was washed with ether, and neutralized with 1N hydrochloric acid. The organic phase was extracted into ethyl acetate, and concentrated. The residue was then purified on a chromatographic column (silica gel 100-200 mesh).

(b) To an aqueous solution of the sodium salt of the acid (prepared by adding 1 equivalent 1N sodium hydroxide solution) was added dropwise an aqueous solution (1M) of calcium acetate (0.55 equiv). White precipitate was obtained, which was filtered off, washed with copious amout of water, and dried in vacuo.

The following compound was prepared following above general procedure

Hemi calcium salt of (3R,5R)-7-[2-(4-Fluorophenyl)-5-isopropyl-3-phenyl-4-(1H-indol-5-yl-amino)carbonyl]-pyrrol-1-yl]-3,5-dihydroxy-heptanoic acid

¹H NMR (DMSO-d₆, 300 MHz): δ 1.21-1.26 (m, 2H), 1.40 (d, J=6 Hz, 6H), 1.42-1.62 (m, 2H), 1.90-1.98 (m, 1H), 2.05-2.12 (m, 1H), 3.19-3.31 (m, 1H), 3.74-3.76 (m, 3H), 3.92-3.96 (m, 1H), 6.33 (s, 1H), 7.00-7.26 (m, 12H), 7.80 (s, 1H), 9.60 (s, 1H), 10.94 (s, 1H); MS (positive ion mode): m/z 598 (Acid+1); Yield: 60%; m.p.: 184-216° C.

Pharmacological Activity

The compounds disclosed herein have activity as inhibitors of 3-hydroxy-3-methyl-glutanyl coenzyme A (HMG-CoA) reductase, and thus are useful in inhibiting cholesterol biosynthesis and/or in lowering triglycerides.

The compounds described herein were screened in an in-vitro HMG-CoA reductase enzyme assay as described by Kubo et al., Endocrinology 120: 214, (1987) and Hellar et al., Biochem and Biophys. Res. Comm. 50: 859, (1973). HMG-CoA reductase is a rate-limiting enzyme in the cholesterol biosynthesis, catalyzing the following reaction: [¹⁴C] HMG-CoA+2NADPH+2H⁺→[¹⁴C] mevanolate+CoA+2NADP⁺ microsomes, utilizing 2.5 μM [¹⁴C] HMG-CoA as a substrate. The reaction was carried out in presence of 100 mM KH₂PO₄, 20 mM G-6-P, 2.5 mM NADP, 10 mM EDTA, 5 mM DTT and 1.4 G-6-P dehydrogenase, at 37° C. for 15 minutes and quantitating [¹⁴C] mevalonate as an end product. For IC₅₀ determination, the compounds dissolved in 1% dimethylsulfoxide were preincubated with liver microsomes at 37° C. for 30 minutes. The IC₅₀ of the compounds of the present invention ranged from about 0.16 nM to about 0.91 nM.

Some of the compounds disclosed herein have intrinsic clearance in human liver microsome significantly less than atorvastatin and are not major substrate for CYP3A4 (cytochrome p450 3A4).

1. A compound having the structure of Formula I, 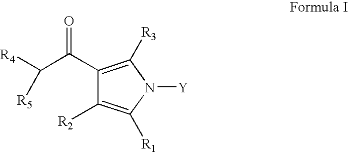
its pharmaceutically acceptable salts, pharmaceutically acceptable solvates, tautomers, racemates, polymorphs, pure enantiomers, diastereoisomers, metabolites, prodrugs or N-oxides wherein 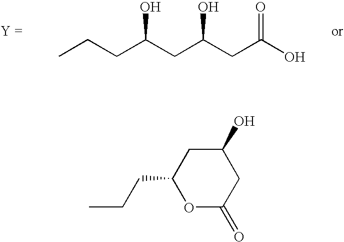
R₁ is C₁-C₆ alkyl, C₃-C₆ cycloalkyl, or optionally substituted phenyl, wherein up to three substituents are independently selected from [halogens, C₁-C₆ alkyl, hydroxyl, C₁-C₃ alkoxy, protected hydroxyl, carboxyl, acetyl, optionally substituted amino wherein up to two substituents are independently selected from C₁-C₆ alkyl, C₃-C₆ cycloalkyl, SO₂R₆, COR₆, CONHR₆ (wherein R₆ is C₁-C₆ alkyl or aryl), C₁-C₃ alkoxycarbonyl, cyano and C₁-C₃ perfluoroalkyl];
R₃ is optionally substituted C₁-C₆ alkyl or C₃-C₆ cycloalkyl (wherein the substituents are selected halogens, hydroxyl, C₁-C₃ alkoxy, and protected hydroxyl); or —NR₇R₈ wherein R₇ and R₈ are optionally substituted C₁-C₆ alkyl (wherein the optional substituent(s) is/are selected from halogens, hydroxyl. C₁-C₃ alkoxy, and protected hydroxyl);
R₂, R₄ and R₅ are independently selected from: hydrogen, C₁-C₆ alkyl, C₃-C₆ cycloalkyl, aralkyl, optionally substituted aryl (wherein the substituents are selected from C₁-C₆ alkyl, C₁-C₆ carbonyl alkyl, C₁-C₆ hydroxyalkyl, halogens, cyano, hydroxyl, protected hydroxyl, C₁-C₆ alkoxy, C₁-C₃ perfluoroalkyl, SO₂NHR₆ (wherein R₆ is C₁-C₆ alkyl, or aryl), COOR₆ wherein R₆ is C₁-C₆ alkyl, or aryl, and —NR₇R₈ wherein R₇ and R₈ are selected from {hydrogen, optionally substituted C₁-C₆ alkyl [wherein the optional substituent(s) is/are selected from halogens, hydroxyl, C₁-C₃ alkoxy, protected hydroxyl, and cyano] optionally substituted C₃-C₆ cycloalkyl [wherein the optional substituent(s) is/are selected from halogens, hydroxyl, C₁-C₃ alkoxy, protected hydroxyl, and cyano], SO₂R₆, COR₆, CONH₂, CONHR₆, COOR₆ [wherein R₆ is C₁-C₆ alkyl or aryl], and optionally substituted aryl [wherein the optional substituent(s) is/are selected from halogens, C₁-C₃ alkyl, hydroxyl, C₁-C₃ alkoxy, protected hydroxyl, and cyano]} and R₂, R₄ and R₅ can also be optionally substituted heterocycle having one or more hetero atom(s) {wherein said hetero atom(s) is/are selected from oxygen, nitrogen and sulfur, and the optional substituents are selected from [optionally substituted C₁-C₆ alkyl or C₃-C₆ cycloalkyl (wherein the optional substituent(s) is/are selected from halogens, hydroxyl, C₁-C₃ alkoxy, protected hydroxyl, and cyano); halogens, hydroxyl, protected hydroxyl, C₁-C₃ alkoxy, cyano, C₁-C₃ perfluoroalkyl, and optionally substituted aryl (wherein the optional substituents are selected from C₁-C₆ alkyl, halogens, hydroxyl, protected hydroxyl, C₁-C₃ alkoxy, cyano, and C₁-C₃ perfluoroalkyl)]},
with the proviso that one of R₂, R₄ and R₅ is a heterocycle and with the further provision that if R₂ is not a heterocycle then either R₄ or R₅ alone is not unsubstituted pyridyl.

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. A compound, which is:
(3R,5R)-7-[2-(4-Fluorophenyl)-5-isopropyl-3-phenyl-4-(4-methylthiazol-2-ylamino)carbonyl]-pyrrol-1-yl]-3,5-dihydroxy-heptanoic acid (Compound No. 1),
(3R,5R)-7-[2-(4-Fluorophenyl)-5-isopropyl-3-(pyridin-2-yl)-4-(phenylamino)carbonyl]-pyrrol-1-yl]-3,5-dihydroxy-heptanoic acid (Compound No. 3),
(3R,5R)-7-[2-(4-Fluorophenyl)-5-isopropyl-3-(pyridin-3-yl)-4-(phenylamino)carbonyl]-pyrrol-1-yl]-3,5-dihydroxy-heptanoic acid (Compound No. 4),
(3R,5R)-7-[2-(4-Fluorophenyl)-5-isopropyl-3-(pyridin-4-yl)-4-(phenylamino)carbonyl]-pyrrol-1-yl]-3,5-dihydroxy-heptanoic acid (Compound No. 5),
(3R,5R)-7-[2-(4-Fluorophenyl)-5-isopropyl-3-(5-methylfuran-2-yl)-4-(phenylamino)carbonyl]-pyrrol-1-yl]-3,5-dihydroxy-heptanoic acid (Compound No. 6),
(3R,5R)-7-[2-(4-Fluorophenyl)-5-isopropyl-3-(thiophen-2-yl)-4-(phenylamino)carbonyl]-pyrrol-1-yl]-3,5-dihydroxy-heptanoic acid (Compound No. 7),
(3R,5R)-7-[2-(4-Fluorophenyl)-5-isopropyl-3-(thiophen-3-yl)-4-(phenylamino)carbonyl]-pyrrol-1-yl]-3,5-dihydroxy-heptanoic acid (Compound No. 8),
(3R,5R)-7-[2-(4-Fluorophenyl)-5-isopropyl-3-phenyl-4-(1H-indol-5-ylamino)carbonyl]-pyrrol-1-yl]-3,5-dihydroxy-heptanoic acid (Compound No. 9),
(3R,5R)-7-[2-(4-fluorophenyl)-5-isopropyl-3-(pyridin-3-yl)-4-(4-acetylphenyl amino)carbonyl]-pyrrol-1-yl]-3,5-dihydroxy-heptanoic acid (compound No. 11),
(3R,5R)-7-[2-(4-fluorophenyl)-5-isopropyl-3-(thiophen-2-yl)-4-(3-fluorophenyl amino)carbonyl]-pyrrol-1-yl]-3,5-dihydroxy-heptanoic acid (compound No. 12),
(3R,5R)-7-[2-(4-fluorophenyl)-5-isopropyl-3-(thiophen-3-yl)-4-(3-fluorophenyl amino)carbonyl]-pyrrol-1-yl]-3,5-dihydroxy-heptanoic acid (compound No. 13),
(3R,5R)-7-[2-(4-fluorophenyl)-5-isopropyl-3-(pyridin-4-yl)-4-(2,4-dimethoxyphenyl amino)carbonyl]-pyrrol-1-yl]-3,5-dihydroxy-heptanoic acid (compound No. 14),
(3R,5R)-7-[2-(4-fluorophenyl)-5-isopropyl-3-(pyridin-3-yl)-4-(2,4-dimethoxyphenyl amino)carbonyl]-pyrrol-1-yl]-3,5-dihydroxy-heptanoic acid (compound No. 15),
(3R,5R)-7-[2-(4-fluorophenyl)-5-isopropyl-3-(pyridin-4-yl)-4-(3-fluorophenyl amino)carbonyl]-pyrrol-1-yl]-3,5-dihydroxy-heptanoic acid (compound No. 16),
(3R,5R)-7-[2-(4-fluorophenyl)-5-isopropyl-3-(pyridin-3-yl)-4-(4-methoxyphenyl amino)carbonyl]-pyrrol-1-yl]-3,5-dihydroxy-heptanoic acid (compound No. 17),
(3R,5R)-7-[2-(4-fluorophenyl)-5-isopropyl-3-(pyridin-3-yl)-4-(3-fluorophenyl amino)carbonyl]-pyrrol-1-yl]-3,5-dihydroxy-heptanoic acid (compound No. 18),
(3R,5R)-7-[2-(4-fluorophenyl)-5-isopropyl-3-(pyridin-3-yl)-4-(2-hydroxyphenyl amino)carbonyl]-pyrrol-1-yl]-3,5-dihydroxy-heptanoic acid (compound No. 19),
(3R,5R)-7-[2-(4-fluorophenyl)-5-isopropyl-3-(pyridin-3-yl)-4-(2-methoxyphenyl amino)carbonyl]-pyrrol-1-yl]-3,5-dihydroxy-heptanoic acid (compound No. 20),
(3R,5R)-7-[2-(4-fluorophenyl)-5-isopropyl-3-(pyridin-4-yl)-4-(4-methoxyphenyl amino)carbonyl]-pyrrol-1-yl]-3,5-dihydroxy-heptanoic acid (compound No. 21),
(3R,5R)-7-[2-(4-fluorophenyl)-5-isopropyl-3-(pyridin-4-yl)-4-(2-hydroxyphenyl amino)carbonyl]-pyrrol-1-yl]-3,5-dihydroxy-heptanoic acid (compound No. 22),
(3R,5R)-7-[2-(4-fluorophenyl)-5-isopropyl-3-(pyridin-4-yl)-4-(2-methoxyphenyl amino)carbonyl]-pyrrol-1-yl]-3,5-dihydroxy-heptanoic acid (compound No. 23),
(3R,5R)-7-[2-(3,4-difluorophenyl)-5-isopropyl-3-(thiophen-3-yl)-4-(phenyl amino)carbonyl]-pyrrol-1-yl]-3,5-dihydroxy-heptanoic acid (compound No. 24),
and their lactone forms, pharmaceutically acceptable salts, pharmaceutically acceptable solvates, tautomers, racemates, polymorphs, pure enantiomers, diastereoisomers, metabolites, prodrugs and N-oxides.

36. A pharmaceutically acceptable salt of a compound of claim 1 which is selected from lithium, sodium, potassium, calcium, magnesium, zinc, aluminium, amino acid, ammonium, mono-alkyl ammonium, dialkyl ammonium, trialkyl ammonium and N-methyl glucamine.

37. (canceled)

38. (canceled)

39. The pharmaceutically acceptable salt of claim 36, wherein the salt is hemicalcium salt.

40. The pharmaceutically acceptable salt of claim 39 wherein the compound is:
Hemi calcium salt of (3R,5R)-7-[2-(4-Fluorophenyl)-5-isopropyl-3-phenyl-4-(4-methylthiazol-2-ylamino)carbonyl]-pyrrol-1-yl]-3,5-dihydroxy-heptanoic acid,
Hemi calcium salt of (3R,5R)-7-[2-(4-Fluorophenyl)-5-isopropyl-3-(pyridin-2-yl)-4-(phenylamino)carbonyl]-pyrrol-1-yl]-3,5-dihydroxy-heptanoic acid,
Hemi calcium salt of (3R,5R)-7-[2-(4-Fluorophenyl)-5-isopropyl-3-(pyridin-3-yl)-4-(phenylamino)carbonyl]-pyrrol-1-yl]-3,5-dihydroxy-heptanoic acid,
Hemi calcium salt of (3R,5R)-7-[2-(4-Fluorophenyl)-5-isopropyl-3-(pyridin-4-yl)-4-(phenylamino)carbonyl]-pyrrol-1-yl]-3,5-dihydroxy-heptanoic acid,
Hemi calcium salt of (3R,5R)-7-[2-(4-Fluorophenyl)-5-isopropyl-3-(5-methylfuran-2-yl)-4-(phenylamino)carbonyl]-pyrrol-1-yl]-3,5-dihydroxy-heptanoic acid,
Hemi calcium salt of (3R,5R)-7-[2-(4-Fluorophenyl)-5-isopropyl-3-(thiophen-2-yl)-4-(phenylamino)carbonyl]-pyrrol-1-yl]-3,5-dihydroxy-heptanoic acid,
Hemi calcium salt of (3R,5R)-7-[2-(4-Fluorophenyl)-5-isopropyl-3-(thiophen-3-yl)-4-(phenylamino)carbonyl]-pyrrol-1-yl]-3,5-dihydroxy-heptanoic acid,
Hemi calcium salt of (3R,5R)-7-[2-(4-Fluorophenyl)-5-isopropyl-3-phenyl-4-(1H-indol-5-yl-amino)carbonyl]-pyrrol-1-yl]-3,5-dihydroxy-heptanoic acid,
Hemi calcium salt of (3R,5R)-7-[2-(4-fluorophenyl)-5-isopropyl-3-(pyridin-3-yl)-4-(4-acetylphenyl amino)carbonyl]-pyrrol-1-yl]-3,5-dihydroxy-heptanoic acid,
Hemi calcium salt of (3R,5R)-7-[2-(4-fluorophenyl)-5-isopropyl-3-(thiophen-2-yl)-4-(3-fluorophenyl amino)carbonyl]-pyrrol-1-yl]-3,5-dihydroxy-heptanoic acid,
Hemi calcium salt of (3R,5R)-7-[2-(4-fluorophenyl)-5-isopropyl-3-(thiophen-3-yl)-4-(3-fluorophenyl amino)carbonyl]-pyrrol-1-yl]-3,5-dihydroxy-heptanoic acid,
Hemi calcium salt of (3R,5R)-7-[2-(4-fluorophenyl)-5-isopropyl-3-(pyridin-4-yl)-4-(2,4-dimethoxyphenyl amino)carbonyl]-pyrrol-1-yl]-3,5-dihydroxy-heptanoic acid,
Hemi calcium salt of (3R,5R)-7-[2-(4-fluorophenyl)-5-isopropyl-3-(pyridin-3-yl)-4-(2,4-dimethoxyphenyl amino)carbonyl]-pyrrol-1-yl]-3,5-dihydroxy-heptanoic acid,
Hemi calcium salt of (3R,5R)-7-[2-(4-fluorophenyl)-5-isopropyl-3-(pyridin-4-yl)-4-(3-fluorophenyl amino)carbonyl]-pyrrol-1-yl]-3,5-dihydroxy-heptanoic acid,
Hemi calcium salt of (3R,5R)-7-[2-(4-fluorophenyl)-5-isopropyl-3-(pyridin-3-yl)-4-(4-methoxyphenyl amino)carbonyl]-pyrrol-1-yl]-3,5-dihydroxy-heptanoic acid,
Hemi calcium salt of (3R,5R)-7-[2-(4-fluorophenyl)-5-isopropyl-3-(pyridin-3-yl)-4-(3-fluorophenyl amino)carbonyl]-pyrrol-1-yl]-3,5-dihydroxy-heptanoic acid,
Hemi calcium salt of (3R,5R)-7-[2-(4-fluorophenyl)-5-isopropyl-3-(pyridin-3-yl)-4-(2-hydroxyphenyl amino)carbonyl]-pyrrol-1-yl]-3,5-dihydroxy-heptanoic acid,
Hemi calcium salt of (3R,5R)-7-[2-(4-fluorophenyl)-5-isopropyl-3-(pyridin-3-yl)-4-(2-methoxyphenyl amino)carbonyl]-pyrrol-1-yl]-3,5-dihydroxy-heptanoic acid,
Hemi calcium salt of (3R,5R)-7-[2-(4-fluorophenyl)-5-isopropyl-3-(pyridin-4-yl)-4-(4-methoxyphenyl amino)carbonyl]-pyrrol-1-yl]-3,5-dihydroxy-heptanoic acid,
Hemi calcium salt of (3R,5R)-7-[2-(4-fluorophenyl)-5-isopropyl-3-(pyridin-4-yl)-4-(2-hydroxyphenyl amino)carbonyl]-pyrrol-1-yl]-3,5-dihydroxy-heptanoic acid),
Hemi calcium salt of (3R,5R)-7-[2-(4-fluorophenyl)-5-isopropyl-3-(pyridin-4-yl)-4-(2-methoxyphenyl amino)carbonyl]-pyrrol-1-yl]-3,5-dihydroxy-heptanoic acid,
Hemi calcium salt of (3R,5R)-7-[2-(3,4-difluorophenyl)-5-isopropyl-3-(thiophen-3-yl)-4-(phenyl amino)carbonyl]-pyrrol-1-yl]-3,5-dihydroxy-heptanoic acid.

41. (canceled)

42. (canceled)

43. (canceled)

44. A pharmaceutical composition comprising a therapeutically effective amount of a compound of claim 1 together with a pharmaceutically acceptable carrier, excipient or diluent.

45. A method for treating a mammal suffering from cholesterol-related disease, diabetes and related disease, cerebrovascular disease or cardiovascular disease, comprising administering to the said mammal, a therapeutically effective amount of a compound of claim 1.

46. A method for treating a mammal suffering from cholesterol-related disease, diabetes and related disease, cerebrovascular disease or cardiovascular disease, comprising administering to the said mammal, a therapeutically effective amount of a pharmaceutical composition according to claim 44.

47. The method according to claim 46 wherein the disease is selected from the group comprising of arteriosclerosis, atherosclerosis, hyperlipidemia, hypercholesterolemia, hypertriglyceridemia, hyperlipoproteinemia, hypertension, stroke, ischemia, endothellium, dysfunctions, peripheral vascular disease, peripheral arterial disease, coronary heart disease, myocardial infarction, cerebral infarction, myocardial microvascular disease, dementia, Alzheimer's disease, osteoporosis and/or osteopenia, angina and restenosis.

48. The method according to claim 47 wherein the disease is hyperlipidemia.

49. The method according to claim 47 wherein the disease is hypercholesterolemia.

50. The method according to claim 47 wherein the disease is hyperlipoproteinemia.

51. The method according to claim 47 wherein the disease is hypertriglyceridemia.

52. The method according to claim 47 wherein the disease is hypertension

53. A process for the preparation of a compound of Formula XI, 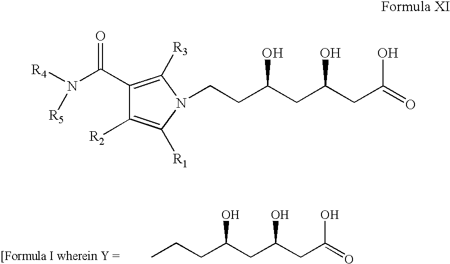
its lactone forms, pharmaceutically acceptable salts, pharmaceutically acceptable solvates, tautomers, racemates, polymorphs, pure enantiomers, diastereoisomers, metabolites, prodrugs or N-oxides wherein
R₁ is C₁-C₆ alkyl, C₃-C₆ cycloalkyl, or optionally substituted phenyl, wherein up to three substituents are independently selected from [halogens, C₁-C₆ alkyl, hydroxyl, C₁-C₃ alkoxy, protected hydroxyl, carboxyl, acetyl, optionally substituted amino wherein up to two substituents are independently selected from C₁-C₆ alkyl, C₃-C₆ cycloalkyl, SO₂R₆, COR₆, CONHR₆ (wherein R₆ is C₁-C₆ alkyl or aryl), C₁-C₃ alkoxycarbonyl, cyano and C₁-C₃ perfluoroalkyl];
R₃ is optionally substituted C₁-C₆ alkyl or C₃-C₆ cycloalkyl (wherein the substituents are selected halogens, hydroxyl, C₁-C₃ alkoxy, and protected hydroxyl); or —NR₇R₈ wherein R₇ and R₈ are optionally substituted C₁-C₆ alkyl (wherein the optional substituent(s) is/are selected from halogens, hydroxyl. C₁-C₃ alkoxy, and protected hydroxyl);
R₂, R₄ and R₅ are independently selected from: hydrogen, C₁-C₆ alkyl, C₃-C₆ cycloalkyl, aralkyl, optionally substituted aryl (wherein the substituents are selected from C₁-C₆ alkyl, C₁-C₆ carbonyl alkyl, C₁-C₆ hydroxyalkyl, halogens, cyano, hydroxyl, protected hydroxyl, C₁-C₆ alkoxy, C₁-C₃ perfluoroalkyl, SO₂NHR₆ (wherein R₆ is C₁-C₆ alky, or aryl), COOR₆ wherein R₆ is C₁-C₆ alkyl, or aryl, and —NR₇R₈ wherein R₇ and R₈ are selected from {hydrogen, optionally substituted C₁-C₆ alkyl [wherein the optional substituent(s) is/are selected from halogens, hydroxyl, C₁-C₃ alkoxy, protected hydroxyl, and cyano] optionally substituted C₃-C₆ cycloalkyl [wherein the optional substituent(s) is/are selected from halogens, hydroxyl, C₁-C₃ alkoxy, protected hydroxyl, and cyano], SO₂R₆, COR₆, CONH₂, CONHR₆, COOR₆ [wherein R₆ is C₁-C₆ alkyl or aryl], and optionally substituted aryl [wherein the optional substituent(s) is/are selected from halogens, C₁-C₃ alkyl, hydroxyl, C₁-C₃ alkoxy, protected hydroxyl, and cyano]} and R₂, R₄ and R₅ can also be optionally substituted heterocycle having one or more hetero atom(s) {wherein said hetero atom(s) is/are selected from oxygen, nitrogen and sulfur, and the optional substituents are selected from [optionally substituted C₁-C₆ alkyl or C₃-C₆ cycloalkyl (wherein the optional substituent(s) is/are selected from halogens, hydroxyl, C₁-C₃ alkoxy, protected hydroxyl, and cyano); halogens, hydroxyl, protected hydroxyl, C₁-C₃ alkoxy, cyano, C₁-C₃ perfluoroalkyl, and optionally substituted aryl (wherein the optional substituents are selected from C₁-C₆ alkyl, halogens, hydroxyl, protected hydroxyl, C₁-C₃ alkoxy, cyano, and C₁-C₃ perfluoroalkyl)]},
with the proviso that one of R₂, R₄ and R₅ is a heterocycle and with the further provision that if R₂ is not a heterocycle then either R₄ or R₅ alone is not unsubstituted pyridyl,
comprising:
reacting a compound of Formula II with a compound of Formula III to give a compound of Formula IV; 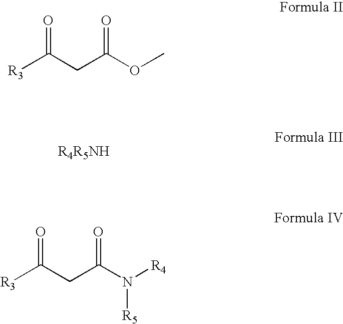
treating the compound of Formula IV with an aldehyde of Formula V to give a compound of Formula VI; 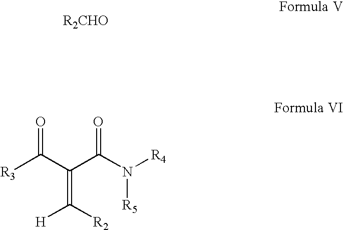
treating the compound of Formula VI with an aldehyde of Formula VII to give a compound of Formula VIII; 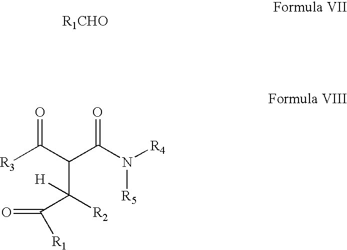
treating the compound of Formula VIII with a compound of Formula IX to give a compound of Formula X, which (when R₄ or R₅ is 2-benzyloxyphenyl) on debenzylation gives a compound of Formula X-A (wherein R₄ or R₅ is 2-hydroxyphenyl); and 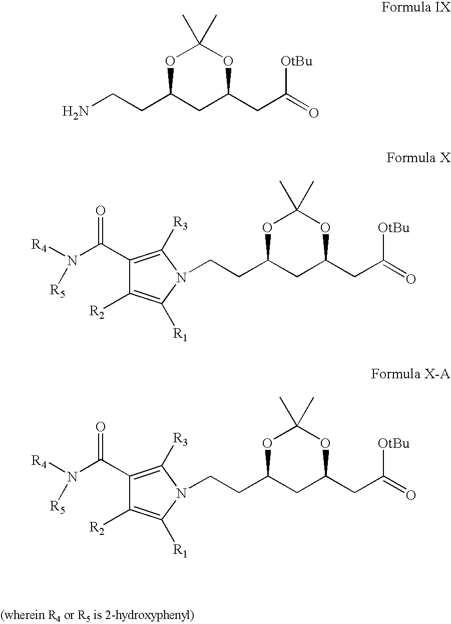
hydrolysing the compound of Formula X or X-A to give a compound of Formula XI.

54. A process for the preparation of compound of Formula XI, 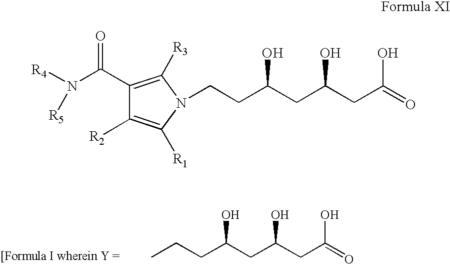
its lactone forms, pharmaceutically acceptable salt, pharmaceutically acceptable solvates, tautomers, racemates, pure enantiomers, prodrugs, metabolites, polymorphs, diastereoisomers or N-oxides wherein
R₁ can be C₁-C₆ alkyl, C₃-C₆ cycloalkyl, or optionally substituted phenyl, wherein up to three substituents are independently selected from [halogens, C₁-C₆ alkyl, hydroxyl, C₁-C₃ alkoxy, protected hydroxyl, carboxyl, acetyl, optionally substituted amino wherein up to two substituents are independently selected from C₁-C₆ alkyl, C₃-C₆ cycloalkyl, SO₂R₆, COR₆, CONHR₆ (wherein R₆ is C₁-C₆ alkyl or aryl), C₁-C₃ alkoxycarbonyl, cyano and C₁-C₃ perfluoroalkyl];
R₃ can be optionally substituted C₁-C₆ alkyl or C₃-C₆ cycloalkyl (wherein the substituents are selected halogens, hydroxyl, C₁-C₃ alkoxy, and protected hydroxyl); or
—NR₇R₈ wherein R₇ and R₈ are optionally substituted C₁-C₆ alkyl (wherein the optional substituent(s) is/are selected from halogens, hydroxyl. C₁-C₃ alkoxy, and protected hydroxyl);
R₂, R₄ and R₅ are independently selected from: hydrogen, C₁-C₆ alkyl, C₃-C₆ cycloalkyl, aralkyl, optionally substituted aryl (wherein the substituents are selected from C₁-C₆ alkyl, C₁-C₆ carbonyl alkyl, C₁-C₆ hydroxyalkyl, halogens, cyano, hydroxyl, protected hydroxyl, C₁-C₆ alkoxy, C₁-C₃ perfluoroalkyl, SO₂NHR₆ (wherein R₆ is C₁-C₆ alky, or aryl), COOR₆ wherein R₆ is C₁-C₆ alkyl, or aryl, and —NR₇R₈ wherein R₇ and R₈ are selected from {hydrogen, optionally substituted C₁-C₆ alkyl [wherein the optional substituent(s) is/are selected from halogens, hydroxyl, C₁-C₃ alkoxy, protected hydroxyl, and cyano] optionally substituted C₃-C₆ cycloalkyl [wherein the optional substituent(s) is/are selected from halogens, hydroxyl, C₁-C₃ alkoxy, protected hydroxyl, and cyano], SO₂R₆, COR₆, CONH₂, CONHR₆, COOR₆ [wherein R₆ is C₁-C₆ alkyl or aryl], and optionally substituted aryl [wherein the optional substituent(s) is/are selected from halogens, C₁-C₃ alkyl, hydroxyl, C₁-C₃ alkoxy, protected hydroxyl, and cyano]} and R₂, R₄ and R₅ can also be optionally substituted heterocycle having one or more hetero atom(s) {wherein said hetero atom(s) is/are selected from oxygen, nitrogen and sulfur, and the optional substituents are selected from [optionally substituted C₁-C₆ alkyl or C₃-C₆ cycloalkyl (wherein the optional substituent(s) is/are selected from halogens, hydroxyl, C₁-C₃ alkoxy, protected hydroxyl, and cyano); halogens, hydroxyl, protected hydroxyl, C₁-C₃ alkoxy, cyano, C₁-C₃ perfluoroalkyl, and optionally substituted aryl (wherein the optional substituents are selected from C₁-C₆ alkyl, halogens, hydroxyl, protected hydroxyl, C₁-C₃ alkoxy, cyano, and C₁-C₃ perfluoroalkyl)]}
with the proviso that one of R₂, R₄ and R₅ is a heterocycle and with the further provision that if R₂ is not a heterocycle then either R₄ or R₅ alone is not unsubstituted pyridyl.
comprising:
reacting a compound of Formula XIII with a compound of Formula V to give a compound of Formula XIV; 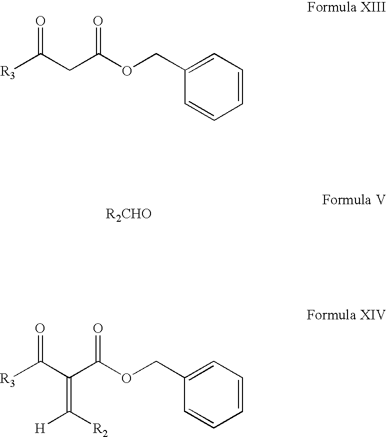
reacting the compound of Formula XIV with a compound of Formula VII to give a compound of Formula XV; 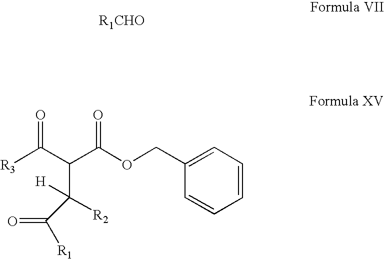
treating the compound of Formula XV with a compound of Formula IX to yield a compound of Formula XVI; 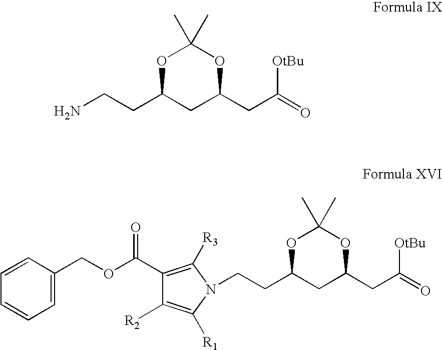
debenzylating the compound of Formula XVI to give a compound of Formula XVII; 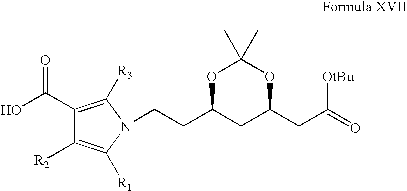
converting the compound of Formula XVII to the corresponding acid chloride; reacting the acid chloride form of the compound of Formula XVII with an amine of Formula III and to give a compound of Formula X; and hydrolyzing the compound of Formula X to give a compound of Formula XI.

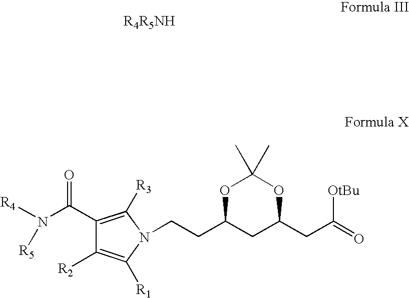

55. A process for the preparation of compound of Formula XI, 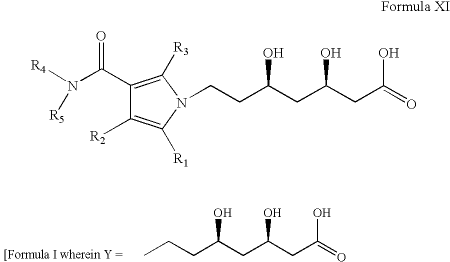
its lactone forms, pharmaceutically acceptable salt, pharmaceutically acceptable solvates, tautomers, racemates, pure enantiomers, prodrugs, metabolites, polymorphs, diastereoisomers or N-oxides wherein
R₁ can be C₁-C₆ alkyl, C₃-C₆ cycloalkyl, or optionally substituted phenyl, wherein up to three substituents are independently selected from [halogens, C₁-C₆ alkyl, hydroxyl, C₁-C₃ alkoxy, protected hydroxyl, carboxyl, acetyl, optionally substituted amino wherein up to two substituents are independently selected from C₁-C₆ alkyl, C₃-C₆ cycloalkyl, SO₂R₆, COR₆, CONHR₆ (wherein R₆ is C₁-C₆ alkyl or aryl), C₁-C₃ alkoxycarbonyl, cyano and C₁-C₃ perfluoroalkyl];
R₃ can be optionally substituted C₁-C₆ alkyl or C₃-C₆ cycloalkyl (wherein the substituents are selected halogens, hydroxyl, C₁-C₃ alkoxy, and protected hydroxyl); or
—NR₇R₈ wherein R₇ and R₈ are optionally substituted C₁-C₆ alkyl (wherein the optional substituent(s) is/are selected from halogens, hydroxyl. C₁-C₃ alkoxy, and protected hydroxyl);
R₂, R₄ and R₅ are independently selected from: hydrogen, C₁-C₆ alkyl, C₃-C₆ cycloalkyl, aralkyl, optionally substituted aryl (wherein the substituents are selected from C₁-C₆ alkyl, C₁-C₆ carbonyl alkyl, C₁-C₆ hydroxyalkyl, halogens, cyano, hydroxyl, protected hydroxyl, C₁-C₆ alkoxy, C₁-C₃ perfluoroalkyl, SO₂NHR₆ (wherein R₆ is C₁-C₆ alky, or aryl), COOR₆ wherein R₆ is C₁-C₆ alkyl, or aryl, and —NR₇R₈ wherein R₇ and R₈ are selected from {hydrogen, optionally substituted C₁-C₆ alkyl [wherein the optional substituent(s) is/are selected from halogens, hydroxyl, C₁-C₃ alkoxy, protected hydroxyl, and cyano]
optionally substituted C₃-C₆ cycloalkyl [wherein the optional substituent(s) is/are selected from halogens, hydroxyl, C₁-C₃ alkoxy, protected hydroxyl, and cyano], SO₂R₆, COR₆, CONH₂, CONHR₆, COOR₆ [wherein R₆ is C₁-C₆ alkyl or aryl], and optionally substituted aryl [wherein the optional substituent(s) is/are selected from halogens, C₁-C₃ alkyl, hydroxyl, C₁-C₃ alkoxy, protected hydroxyl, and cyano]} and R₂, R₄ and R₅ can also be optionally substituted heterocycle having one or more hetero atom(s) {wherein said hetero atom(s) is/are selected from oxygen, nitrogen and sulfur, and the optional substituents are selected from [optionally substituted C₁-C₆ alkyl or C₃-C₆ cycloalkyl (wherein the optional substituent(s) is/are selected from halogens, hydroxyl, C₁-C₃ alkoxy, protected hydroxyl, and cyano); halogens, hydroxyl, protected hydroxyl, C₁-C₃ alkoxy, cyano, C₁-C₃ perfluoroalkyl, and optionally substituted aryl (wherein the optional substituents are selected from C₁-C₆ alkyl, halogens, hydroxyl, protected hydroxyl, C₁-C₃ alkoxy, cyano, and C₁-C₃ perfluoroalkyl)]}
with the proviso that one of R₂, R₄ and R₅ is a heterocycle and with the further provision that if R₂ is not a heterocycle then either R₄ or R₅ alone is not unsubstituted pyridyl.
comprising:
reacting a compound of Formula XIII with a compound of Formula V to give a compound of Formula XIV; 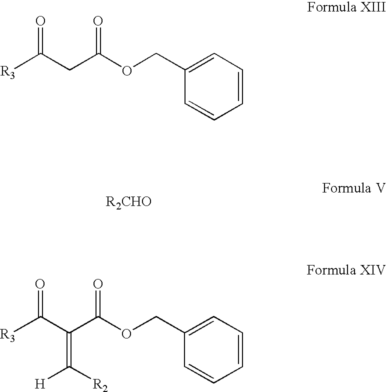
reacting the compound of Formula XIV with a compound of Formula VII to give a compound of Formula XV; 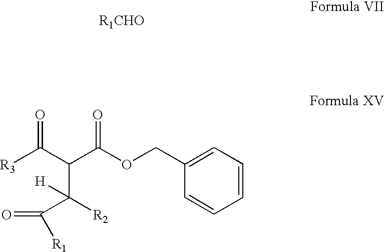
treating the compound of Formula XV with a compound of Formula IX to yield a compound of Formula XVI; 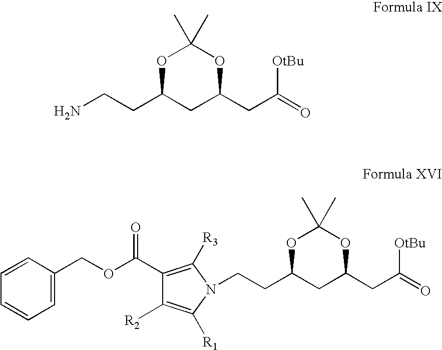
debenzylating the compound of Formula XVI to give a compound of Formula XVII; 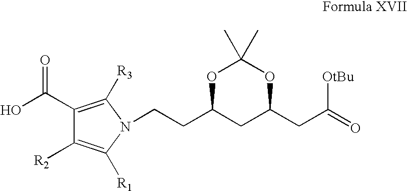
reacting the compound of Formula XVII with an amine of Formula III and a coupling agent to give a compound of Formula X, and hydrolysing the compound of Formula X to give a compound of Formula XI.

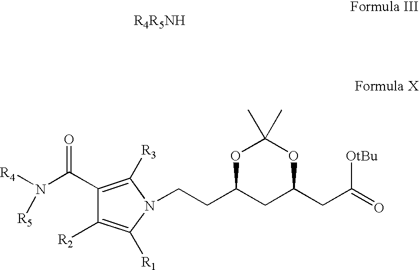

56. A process for the preparation of a compound of Formula XII, 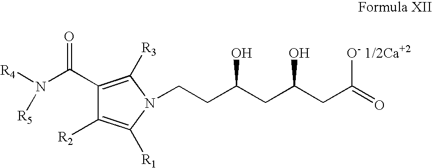
its pharmaceutically acceptable solvates, tautomers, racemates, polymorphs, pure enantiomers, diastereoisomers, metabolites, prodrugs or N-oxides wherein
R₁ is C₁-C₆ alkyl, C₃-C₆ cycloalkyl, or optionally substituted phenyl, wherein up to three substituents are independently selected from [halogens, C₁-C₆ alkyl, hydroxyl, C₁-C₃ alkoxy, protected hydroxyl, carboxyl, acetyl, optionally substituted amino wherein up to two substituents are independently selected from C₁-C₆ alkyl, C₃-C₆ cycloalkyl, SO₂R₆, COR₆, CONHR₆ (wherein R₆ is C₁-C₆ alkyl or aryl), C₁-C₃ alkoxycarbonyl, cyano and C₁-C₃ perfluoroalkyl];
R₃ is optionally substituted C₁-C₆ alkyl or C₃-C₆ cycloalkyl (wherein the substituents are selected halogens, hydroxyl, C₁-C₃ alkoxy, and protected hydroxyl); or —NR₇R₈ wherein R₇ and R₈ are optionally substituted C₁-C₆ alkyl (wherein the optional substituent(s) is/are selected from halogens, hydroxyl. C₁-C₃ alkoxy, and protected hydroxyl);
R₂, R₄ and R₅ are independently selected from: hydrogen, C₁-C₆ alkyl, C₃-C₆ cycloalkyl, aralkyl, optionally substituted aryl (wherein the substituents are selected from C₁-C₆ alkyl, C₁-C₆ carbonyl alkyl, C₁-C₆ hydroxyalkyl, halogens, cyano, hydroxyl, protected hydroxyl, C₁-C₆ alkoxy, C₁-C₃ perfluoroalkyl, SO₂NHR₆ (wherein R₆ is C₁-C₆ alky, or aryl), COOR₆ wherein R₆ is C₁-C₆ alkyl, or aryl, and —NR₇R₈ wherein R₇ and R₈ are selected from {hydrogen, optionally substituted C₁-C₆ alkyl [wherein the optional substituent(s) is/are selected from halogens, hydroxyl, C₁-C₃ alkoxy, protected hydroxyl, and cyano] optionally substituted C₃-C₆ cycloalkyl [wherein the optional substituent(s) is/are selected from halogens, hydroxyl, C₁-C₃ alkoxy, protected hydroxyl, and cyano], SO₂R₆, COR₆, CONH₂, CONHR₆, COOR₆ [wherein R₆ is C₁-C₆ alkyl or aryl], and optionally substituted aryl [wherein the optional substituent(s) is/are selected from halogens, C₁-C₃ alkyl, hydroxyl, C₁-C₃ alkoxy, protected hydroxyl, and cyano]} and R₂, R₄ and R₅ can also be optionally substituted heterocycle having one or more hetero atom(s) {wherein said hetero atom(s) is/are selected from oxygen, nitrogen and sulfur, and the optional substituents are selected from [optionally substituted C₁-C₆ alkyl or C₃-C₆ cycloalkyl (wherein the optional substituent(s) is/are selected from halogens, hydroxyl, C₁-C₃ alkoxy, protected hydroxyl, and cyano); halogens, hydroxyl, protected hydroxyl, C₁-C₃ alkoxy, cyano, C₁-C₃ perfluoroalkyl, and optionally substituted aryl (wherein the optional substituents are selected from C₁-C₆ alkyl, halogens, hydroxyl, protected hydroxyl, C₁-C₃ alkoxy, cyano, and C₁-C₃ perfluoroalkyl)]},
with the proviso that one of R₂, R₄ and R₅ is a heterocycle and with the further provision that if R₂ is not a heterocycle then either R₄ or R₅ alone is not unsubstituted pyridyl,
comprising:
reacting a compound of Formula II with a compound of Formula III to give a compound of Formula IV; 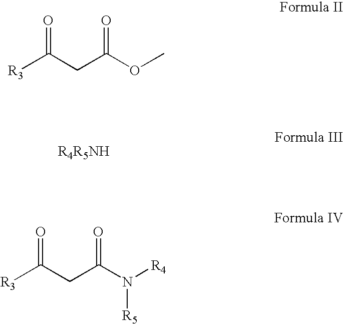
treating the compound of Formula IV with an aldehyde of Formula V to give a compound of Formula VI; 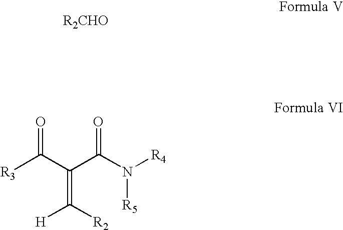
treating the compound of Formula VI with an aldehyde of Formula VII to give a compound of Formula VIII; 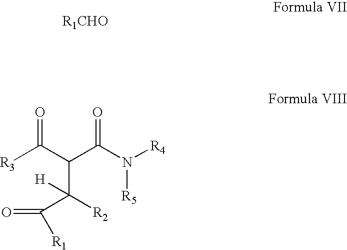
treating the compound of Formula VIII with a compound of Formula IX to give a compound of Formula X, which (when R₄ or R₅ is 2-benzyloxyphenyl) on debenzylation gives a compound of Formula X-A (wherein R₄ or R₅ is 2-hydroxyphenyl); and 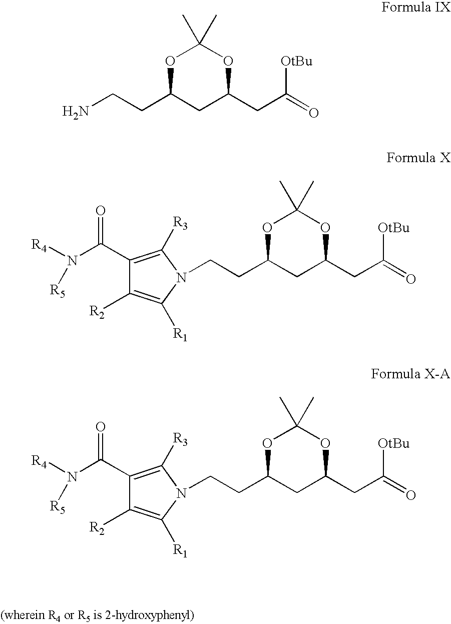
hydrolysing the compound of Formula X or X-A to give a compound of Formula XI, to give a compound of Formula XI; 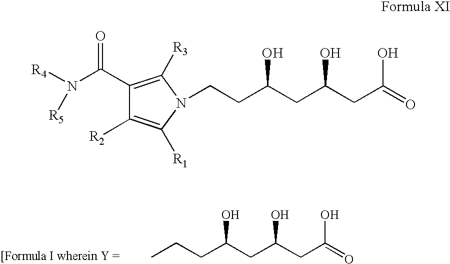
treating the compound of Formula XI with sodium hydroxide followed by calcium acetate to give the hemi calcium salt of Formula XII.

57. A process for the preparation of compound of Formula XII, 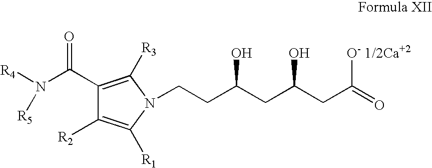
its lactone forms, pharmaceutically acceptable salt, pharmaceutically acceptable solvates, tautomers, racemates, polymorphs, prodrugs, metabolites, pure enantiomers, diastereoisomers or N-oxides wherein
R₁ is C₁-C₆ alkyl, C₃-C₆ cycloalkyl, or optionally substituted phenyl, wherein up to three substituents are independently selected from [halogens, C₁-C₆ alkyl, hydroxyl, C₁-C₃ alkoxy, protected hydroxyl, carboxyl, acetyl, optionally substituted amino wherein up to two substituents are independently selected from C₁-C₆ alkyl, C₃-C₆ cycloalkyl, SO₂R₆, COR₆, CONHR₆ (wherein R₆ is C₁-C₆ alkyl or aryl), C₁-C₃ alkoxycarbonyl, cyano and C₁-C₃ perfluoroalkyl];
R₃ is optionally substituted C₁-C₆ alkyl or C₃-C₆ cycloalkyl (wherein the substituents are selected halogens, hydroxyl, C₁-C₃ alkoxy, and protected hydroxyl); or —NR₇R₈ wherein R₇ and R₈ are optionally substituted C₁-C₆ alkyl (wherein the optional substituent(s) is/are selected from halogens, hydroxyl. C₁-C₃ alkoxy, and protected hydroxyl);
R₂, R₄ and R₅ are independently selected from: hydrogen, C₁-C₆ alkyl, C₃-C₆ cycloalkyl, aralkyl, optionally substituted aryl (wherein the substituents are selected from C₁-C₆ alkyl, C₁-C₆ carbonyl alkyl, C₁-C₆ hydroxyalkyl, halogens, cyano, hydroxyl, protected hydroxyl, C₁-C₆ alkoxy, C₁-C₃ perfluoroalkyl, SO₂NHR₆ (wherein R₆ is C₁-C₆ alky, or aryl), COOR₆ wherein R₆ is C₁-C₆ alkyl, or aryl, and —NR₇R₈ wherein R₇ and R₈ are selected from {hydrogen, optionally substituted C₁-C₆ alkyl [wherein the optional substituent(s) is/are selected from halogens, hydroxyl, C₁-C₃ alkoxy, protected hydroxyl, and cyano] optionally substituted C₃-C₆ cycloalkyl [wherein the optional substituent(s) is/are selected from halogens, hydroxyl, C₁-C₃ alkoxy, protected hydroxyl, and cyano], SO₂R₆, COR₆, CONH₂, CONHR₆, COOR₆ [wherein R₆ is C₁-C₆ alkyl or aryl], and optionally substituted aryl [wherein the optional substituent(s) is/are selected from halogens, C₁-C₃ alkyl, hydroxyl, C₁-C₃ alkoxy, protected hydroxyl, and cyano]} and R₂, R₄ and R₅ can also be optionally substituted heterocycle having one or more hetero atom(s) {wherein said hetero atom(s) is/are selected from oxygen, nitrogen and sulfur, and the optional substituents are selected from [optionally substituted C₁-C₆ alkyl or C₃-C₆ cycloalkyl (wherein the optional substituent(s) is/are selected from halogens, hydroxyl, C₁-C₃ alkoxy, protected hydroxyl, and cyano); halogens, hydroxyl, protected hydroxyl, C₁-C₃ alkoxy, cyano, C₁-C₃ perfluoroalkyl, and optionally substituted aryl (wherein the optional substituents are selected from C₁-C₆ alkyl, halogens, hydroxyl, protected hydroxyl, C₁-C₃ alkoxy, cyano, and C₁-C₃ perfluoroalkyl)]},
with the proviso that one of R₂, R₄ and R₅ is a heterocycle and with the further provision that if R₂ is not a heterocycle then either R₄ or R₅ alone is not unsubstituted pyridyl,
comprising:
reacting a compound of Formula XIII with a compound of Formula V to give a compound of Formula XIV; 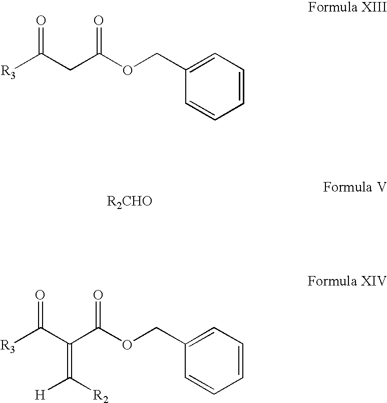
reacting the compound of Formula XIV with a compound of Formula VII to give a compound of Formula XV; 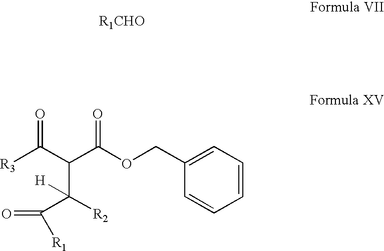
treating the compound of Formula XV with a compound of Formula IX to yield a compound of Formula XVI; 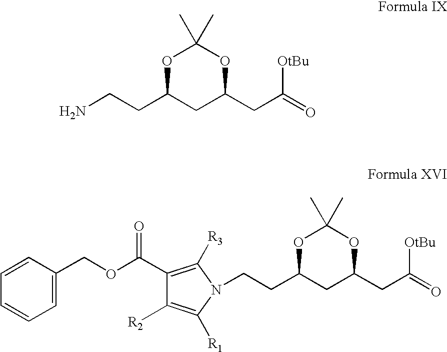
debenzylating the compound of Formula XVI to give a compound of Formula XVII; 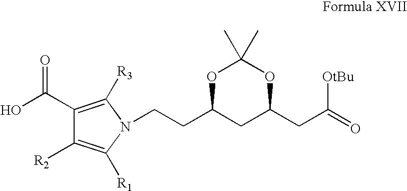
converting the compound of Formula XVII to the corresponding acid chloride; reacting the acid chloride form of the compound of Formula XVII with an amine of Formula III and to give a compound of Formula X; and hydrolyzing the compound of Formula X 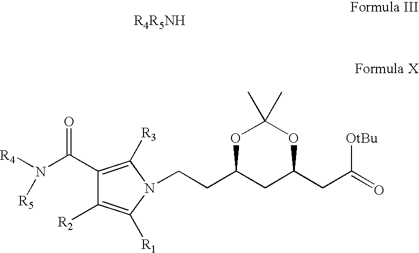
to give a compound of Formula XI; 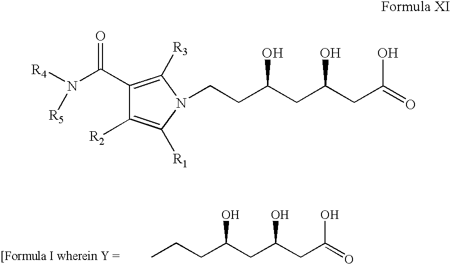
treating the compound of Formula XI with sodium hydroxide followed by calcium acetate to give the hemi calcium salt of Formula XII.

58. A process for the preparation of a compound of Formula XII, 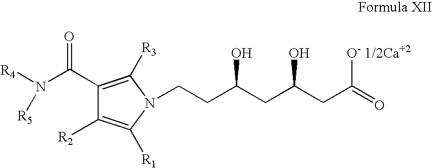
its pharmaceutically acceptable solvates, tautomers, racemates, polymorphs, prodrugs, metabolites, pure enantiomers, diastereoisomers or N-oxides wherein
R₁ is C₁-C₆ alkyl, C₃-C₆ cycloalkyl, or optionally substituted phenyl, wherein up to three substituents are independently selected from [halogens, C₁-C₆ alkyl, hydroxyl, C₁-C₃ alkoxy, protected hydroxyl, carboxyl, acetyl, optionally substituted amino wherein up to two substituents are independently selected from C₁-C₆ alkyl, C₃-C₆ cycloalkyl, SO₂R₆, COR₆, CONHR₆ (wherein R₆ is C₁-C₆ alkyl or aryl), C₁-C₃ alkoxycarbonyl, cyano and C₁-C₃ perfluoroalkyl];
R₃ is optionally substituted C₁-C₆ alkyl or C₃-C₆ cycloalkyl (wherein the substituents are selected halogens, hydroxyl, C₁-C₃ alkoxy, and protected hydroxyl); or —NR₇R₈ wherein R₇ and R₈ are optionally substituted C₁-C₆ alkyl (wherein the optional substituent(s) is/are selected from halogens, hydroxyl. C₁-C₃ alkoxy, and protected hydroxyl);
R₂, R₄ and R₅ are independently selected from: hydrogen, C₁-C₆ alkyl, C₃-C₆ cycloalkyl, aralkyl, optionally substituted aryl (wherein the substituents are selected from C₁-C₆ alkyl, C₁-C₆ carbonyl alkyl, C₁-C₆ hydroxyalkyl, halogens, cyano, hydroxyl, protected hydroxyl, C₁-C₆ alkoxy, C₁-C₃ perfluoroalkyl, SO₂NHR₆ (wherein R₆ is C₁-C₆ alky, or aryl), COOR₆ wherein R₆ is C₁-C₆ alkyl, or aryl, and —NR₇R₈ wherein R₇ and R₈ are selected from {hydrogen, optionally substituted C₁-C₆ alkyl [wherein the optional substituent(s) is/are selected from halogens, hydroxyl, C₁-C₃ alkoxy, protected hydroxyl, and cyano] optionally substituted C₃-C₆ cycloalkyl [wherein the optional substituent(s) is/are selected from halogens, hydroxyl, C₁-C₃ alkoxy, protected hydroxyl, and cyano], SO₂R₆, COR₆, CONH₂, CONHR₆, COOR₆ [wherein R₆ is C₁-C₆ alkyl or aryl], and optionally substituted aryl [wherein the optional substituent(s) is/are selected from halogens, C₁-C₃ alkyl, hydroxyl, C₁-C₃ alkoxy, protected hydroxyl, and cyano]} and R₂, R₄ and R₅ can also be optionally substituted heterocycle having one or more hetero atom(s) {wherein said hetero atom(s) is/are selected from oxygen, nitrogen and sulfur, and the optional substituents are selected from [optionally substituted C₁-C₆ alkyl or C₃-C₆ cycloalkyl (wherein the optional substituent(s) is/are selected from halogens, hydroxyl, C₁-C₃ alkoxy, protected hydroxyl, and cyano); halogens, hydroxyl, protected hydroxyl, C₁-C₃ alkoxy, cyano, C₁-C₃ perfluoroalkyl, and optionally substituted aryl (wherein the optional substituents are selected from C₁-C₆ alkyl, halogens, hydroxyl, protected hydroxyl, C₁-C₃ alkoxy, cyano, and C₁-C₃ perfluoroalkyl)]},
with the proviso that one of R₂, R₄ and R₅ is a heterocycle and with the further provision that if R₂ is not a heterocycle then either R₄ or R₅ alone is not unsubstituted pyridyl,
comprising:
reacting a compound of Formula XIII with a compound of Formula V to give a compound of Formula XIV; 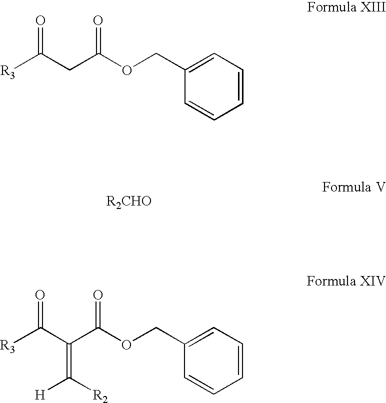
reacting the compound of Formula XIV with a compound of Formula VII to give a compound of Formula XV; 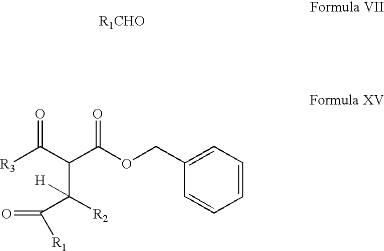
treating the compound of Formula XV with a compound of Formula IX to yield a compound of Formula XVI; 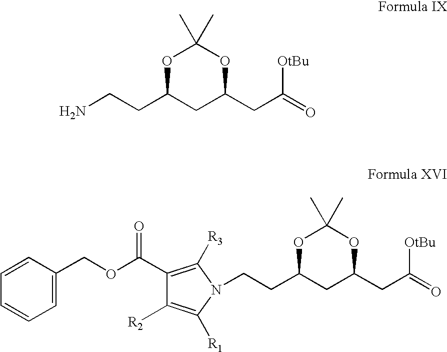
debenzylating the compound of Formula XVI to give a compound of Formula XVII; 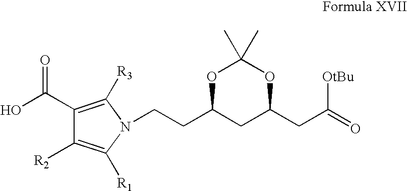
reacting the compound of Formula XVII with an amine of Formula III and a coupling agent to give a compound of Formula X; and hydrolyzing the compound of Formula X, 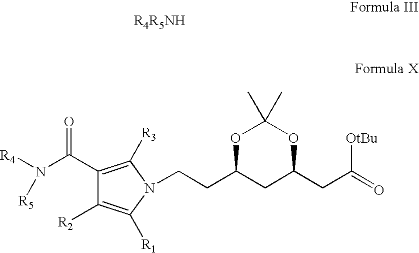
to give a compound of Formula XI; 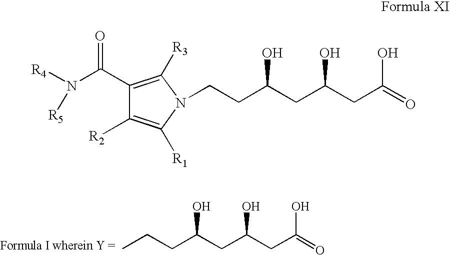
treating the compound of Formula XI with sodium hydroxide followed by calcium acetate to give the hemi calcium salt of Formula XII.
